(19) United States
(12) Patent Application Publication
(10) Pub. No.: US 2023/0193182 A1
(43) Pub. Date: Jun. 22, 2023

(54) MULTIPLANAR MICROFLUIDIC DEVICES WITH MULTIDIRECTIONAL DIRECT FLUID COMMUNICATION AMONG ADJACENT MICROFLUIDIC CHANNELS

(71) Applicant: THE GOVERNING COUNCIL OF THE UNIVERSITY OF TORONTO, Toronto (CA)

(72) Inventors: Edmond YOUNG, Toronto (CA); Michael MOHAN, Toronto (CA)

(21) Appl. No.: 17/953,949

(22) Filed: Sep. 27, 2022

Related U.S. Application Data
(60) Provisional application No. 63/248,720, filed on Sep. 27, 2021.

(51) Int. Cl. C12M 1/00; C12M 3/00; C12M 3/06; C12M 1/12; B01L 3/00; C12N 5/00

(52) U.S. Cl. CPC C12M 29/10; C12M 21/08; C12M 23/16; C12M 25/14; B01L 3/502715; C12N 5/0062; C12N 2533/52; C12N 2533/54; C12N 2513/00; B01L 2300/0874; B01L 2300/0822

(57) ABSTRACT

Multiplanar microfluidic devices are provided that facilitate direct transverse fluid communication between a first microfluidic channel a plurality of adjacent microfluidic channels, where the adjacent microfluidic channels reside both laterally adjacent and vertically adjacent to the first microfluidic channel, thereby facilitating transverse diffusion to or from the adjacent microfluidic channels in both lateral and vertical directions. Geometrical meniscus-pinning features, such as meniscus-pinning ridge structures, are provided between adjacent microfluidic channels to restrict transverse flow between the microfluidic channels. Accordingly, a gel structure may be formed within the first microfluidic channel and one or more of the adjacent microfluidic channels can function as a perfusion channel, for example, for delivering media to cells residing withing the gel structure. Such devices may be extended and/or arrayed to include multiple channels with laterally and vertically adjacent perfusion microfluidic channels, optionally with shared lateral perfusion microfluidic channels among adjacent pairs of devices.

### CROSS-REFERENCE TO RELATED APPLICATION

This application claims priority to U.S. Provisional Patent Application No. 63/248,720, titled “MULTIPLANAR MICROFLUIDIC DEVICES WITH MULTIDIRECTIONAL DIRECT FLUID COMMUNICATION AMONG ADJACENT MICROFLUIDIC CHANNELS” and filed on Sep. 27, 2021, the entire contents of which is incorporated herein by reference.

### BACKGROUND

The present disclosure relates to microfluidic devices. More particularly, the present disclosure relates to biomicrofluidic and organ-on-a-chip devices.

Biomicrofluidic devices systems can be employed to facilitate the in vitro organization of living cells, extracellular matrix (ECM) components, biomaterials, and other elements in microfluidic geometries in such a manner that enables physiological mimicry of three-dimensional (3D) tissue microenvironments. These engineered systems are significant advances to the in vitro tools currently used in biology and biomedical research because they possess several major advantages over traditional in vitro platforms and enable organ-on-a-chip devices. First, biomicrofluidic devices can offer precise spatiotemporal control of the 3D microenvironment, which cannot be achieved by traditional 2D or 3D hydrogel-based cell cultures. Second, biomicrofluidic devices allow application of multiple physical, biochemical, and mechanical cues in combination to create more complex microenvironments. Third, biomicrofluidic devices—and microfluidic systems in general—can manipulate small volumes of fluids and handle small sample sizes, and can be reduced in footprint to increase throughput. Taken together, these advantages have made biomicrofluidic devices promising technologies for basic research, clinical studies, and drug development.

### SUMMARY

Multiplanar microfluidic devices are provided that facilitate direct transverse fluid communication between a first microfluidic channel and a plurality of adjacent microfluidic channels, where the adjacent microfluidic channels reside both laterally adjacent and vertically adjacent to the first microfluidic channel, thereby facilitating transverse diffusion to or from the adjacent microfluidic channels in both lateral and vertical directions. Geometrical meniscus-pinning features, such as meniscus-pinning ridge structures, are provided between adjacent microfluidic channels to restrict transverse flow between the microfluidic channels. Accordingly, a gel structure may be formed within the first microfluidic channel and one or more of the adjacent microfluidic channels can function as a perfusion channel, for example, for delivering media to cells residing withing the gel structure. Such devices may be extended and/or arrayed to include multiple channels with laterally and vertically adjacent perfusion microfluidic channels, optionally with shared lateral perfusion microfluidic channels among adjacent pairs of devices.

Accordingly, in a first aspect, there is provided a microfluidic device comprising:
a first microfluidic channel extending within a first horizontal planar region;
a second microfluidic channel extending within the first horizontal planar region, the second microfluidic channel residing laterally adjacent to the first microfluidic channel along a first portion of the first microfluidic channel such that the second microfluidic channel is in direct lateral fluid communication with the first microfluidic channel along the first portion of the first microfluidic channel in the absence of a membrane therebetween;
a third microfluidic channel extending within a second horizontal planar region, the second horizontal planar region being vertically offset from the first horizontal planar region, the third microfluidic channel residing vertically adjacent to the first microfluidic channel along a second portion of the first microfluidic channel such that the third microfluidic channel is in direct vertical fluid communication with the first microfluidic channel along the second portion of the first microfluidic channel in the absence of a membrane therebetween;
a lateral geometrical meniscus-pinning feature residing between the second microfluidic channel and the first microfluidic channel within the first portion of the first microfluidic channel, the lateral geometrical meniscus-pinning feature being configured to resist fluid flow between the first microfluidic channel and the second microfluidic channel within the first portion of the first microfluidic channel; and
a vertical geometrical meniscus-pinning feature residing between the third microfluidic channel and the first microfluidic channel within the second portion of the first microfluidic channel, the vertical geometrical meniscus-pinning feature being configured to resist fluid flow between the first microfluidic channel and the third microfluidic channel within the second portion of the first microfluidic channel.

In some example implementations of the device, the first portion of the first microfluidic channel overlaps at least in part with the second portion of the first microfluidic channel.

In some example implementations of the device, at least one of the lateral geometrical meniscus-pinning feature and the vertical geometrical meniscus-pinning feature comprises an elongate meniscus-pinning edge feature extending longitudinally between the first portion of the first microfluidic channel and the second microfluidic channel. The elongate meniscus-pinning edge feature may include a meniscus-pinning ridge.

In some example implementations of the device, the lateral geometrical meniscus-pinning feature comprises a lateral meniscus-pinning ridge and the vertical geometrical meniscus-pinning feature comprises a vertical meniscus-pinning ridge, the lateral meniscus-pinning ridge and the vertical meniscus-pinning ridge being configured to resist fluid flow in orthogonal directions.

In some example implementations of the device, at least one of the lateral geometrical meniscus-pinning feature and the vertical geometrical meniscus-pinning feature comprises a plurality of microposts.

In some example implementations, the device further includes a first inlet port and a first outlet port in flow communication with the first microfluidic channel. The first inlet port may be configured to be sealed by insertion of a pipette tip, thereby facilitating dispensing of a fluid into the first microfluidic channel from the pipette tip after insertion of the pipette tip into the first inlet port. The device may further include a second inlet port and a second outlet port in flow communication with the second microfluidic channel; and a third inlet port and a third outlet port in flow communication with the third microfluidic channel; wherein the second inlet port, the second outlet port, the third inlet port and the third outlet port reside within respective microwells.

In some example implementations, the device further includes:
a first layer;
a second layer bonded to the first layer;
wherein at least one of an upper surface of the first layer and a lower surface of the second layer comprise recessed features that define, after the first layer is bonded to the second layer, the first microfluidic channel, the second microfluidic channel, and the lateral geometrical meniscus-pinning feature;
the second layer further comprising at least one aperture defined such that the aperture resides above the first microfluidic channel after the first layer is bonded to the second layer; and
a third layer secured to the second layer, wherein a lower surface of the third layer comprises an additional recessed feature that defines, after the third layer is bonded to the second layer, the third microfluidic channel, and wherein the aperture forms, at least in part, the vertical geometrical meniscus-pinning feature. The third layer may be detachably removable from the second layer to provide direct access to contents of the first microfluidic channel.

In some example implementations, the device further includes a reservoir defined above at least a portion of the third microfluidic channel, the reservoir being in fluid communication with the third microfluidic channel via a horizontal membrane residing between the reservoir and the third microfluidic channel.

In some example implementations of the device, the lateral geometrical meniscus-pinning feature is a first lateral geometrical meniscus-pinning feature, and the microfluidic device further includes:
a fourth microfluidic channel extending within the first horizontal planar region, the fourth microfluidic channel residing laterally adjacent to the first microfluidic channel along a third portion of the first microfluidic channel such that the fourth microfluidic channel is in direct lateral fluid communication with the first microfluidic channel along the third portion of the first microfluidic channel in the absence of a membrane therebetween, the first microfluidic channel residing between the second microfluidic channel and the fourth microfluidic channel; and
a second lateral geometrical meniscus-pinning feature residing between the first microfluidic channel and the fourth microfluidic channel within the third portion of the first microfluidic channel, the second lateral geometrical meniscus-pinning feature being configured to resist fluid flow between the first microfluidic channel and the fourth microfluidic channel within the third portion of the first microfluidic channel.

The vertical geometrical meniscus-pinning feature may be a first vertical geometrical meniscus-pinning feature, and the microfluidic device may further include:
a fifth microfluidic channel extending within the first horizontal planar region, the fifth microfluidic channel residing laterally adjacent to the fourth microfluidic channel along a first portion of the fifth microfluidic channel, such that the fourth microfluidic channel is in direct lateral fluid communication with the fifth microfluidic channel along the first portion of the fifth microfluidic channel in the absence of a membrane therebetween, the fourth microfluidic channel residing between the first microfluidic channel and the fifth microfluidic channel;
a third lateral geometrical meniscus-pinning feature residing between the fourth microfluidic channel and the fifth microfluidic channel within the first portion of the fifth microfluidic channel, the third lateral geometrical meniscus-pinning feature being configured to resist fluid flow between the fifth microfluidic channel and the fourth microfluidic channel within the first portion of the fifth microfluidic channel;
a sixth microfluidic channel extending within the second horizontal planar region, the sixth microfluidic channel residing vertically adjacent to the fifth microfluidic channel along a second portion of the fifth microfluidic channel such that the sixth microfluidic channel is in direct vertical fluid communication with the fifth microfluidic channel along the second portion of the fifth microfluidic channel in the absence of a membrane therebetween; and
a second vertical geometrical meniscus-pinning feature residing between the sixth microfluidic channel and the fifth microfluidic channel within the second portion of the fifth microfluidic channel, the vertical geometrical meniscus-pinning feature being configured to resist fluid flow between the fifth microfluidic channel and the sixth microfluidic channel within the second portion of the fifth microfluidic channel.

The first portion of the fifth microfluidic channel may overlap at least in part with the second portion of the fifth microfluidic channel.

The device may further include: a seventh microfluidic channel extending within the first horizontal planar region, the seventh microfluidic channel residing laterally adjacent to the fifth microfluidic channel along a third portion of the fifth microfluidic channel, such that the seventh microfluidic channel is in direct lateral fluid communication with the fifth microfluidic channel along the third portion of the fifth microfluidic channel in the absence of a membrane therebetween, the fifth microfluidic channel residing between the fourth microfluidic channel and the seventh microfluidic channel; and a fourth lateral geometrical meniscus-pinning feature residing between the seventh microfluidic channel and the fifth microfluidic channel within the third portion of the fifth microfluidic channel, the fourth lateral geometrical meniscus-pinning feature being configured to resist fluid flow between the fifth microfluidic channel and the seventh microfluidic channel within the third portion of the fifth microfluidic channel.

In some example implementations of the device, a substrate of the microfluidic device comprises a planar surface residing below the first horizontal planar region, such that the planar surface and the second horizontal planar region reside on opposite sides of the first horizontal planar region, and wherein a portion of the substrate residing between the planar surface and the first microfluidic channel is sufficiently transparent to permit microscopic imaging of at least the first microfluidic channel.

In some example implementations of the device, the first microfluidic channel comprises a gel, and wherein the second microfluidic channel and the third microfluidic channel are substantially absent of the gel.

In some example implementations of the device, the second microfluidic channel and the third microfluidic channel each comprise a gel, and wherein the first microfluidic channel is substantially absent of the gel.

In another aspect, there is provided a plurality of microfluidic devices as described above, wherein each microfluidic device is defined within a common substrate. The plurality of microfluidic devices may be defined in an arrayed format on a fluidic chip. The fluidic chip may have a length of 75 mm plus or minus 2 mm and a width of 26 mm plus or minus 2 mm, such that lateral dimensions of the fluidic chip are substantially equivalent to those of a conventional microscope slide. The plurality of microfluidic devices may be defined in an arrayed format on a microplate.

In another aspect, there is provided a microfluidic apparatus comprising:
a plurality of microfluidic devices as described above, wherein each microfluidic device is defined within a common substrate; and
wherein, for at least one adjacent pair of microfluidic devices, the adjacent pair of microfluidic devices comprising a first microfluidic device and a second microfluidic device:
   the first microfluidic channel of the first microfluidic device resides laterally adjacent to the second microfluidic channel of the second microfluidic device along an additional portion of the first microfluidic channel, such that the second microfluidic channel of the second microfluidic device is in direct lateral fluid communication with the first microfluidic channel of the first microfluidic device along the first portion of the first microfluidic channel of the first microfluidic device in the absence of a membrane therebetween; and
   an additional lateral geometrical meniscus-pinning feature resides between the second microfluidic channel of the second microfluidic device and the first microfluidic channel of the first microfluidic device within the additional portion of the first microfluidic channel, the lateral geometrical meniscus-pinning feature being configured to resist fluid flow between the first microfluidic channel of the first microfluidic device and the second microfluidic channel of the second microfluidic device within the additional portion of the first microfluidic channel of the first microfluidic device.

The plurality of microfluidic devices may be defined in an arrayed format on a fluidic chip. The fluidic chip may have a length of 75 plus or minus 2 mm and a width of 26 plus or minus 2 mm, such that lateral dimensions of the fluidic chip are substantially equivalent to those of a conventional microscope slide. The plurality of microfluidic devices may be defined in an arrayed format on a microplate.

In another aspect, there is provided a method of performing microfluidic perfusion of a cell-containing structure, the method comprising:
providing a microfluidic device as described above;
forming the cell-containing structure within the first microfluidic channel such that the cell-containing structure contains viable cells; and
injecting a perfusion liquid into one or both of the second microfluidic channel and the third microfluidic channel, the perfusion liquid comprising growth media; and
incubating the microfluidic device while contacting the perfusion liquid with the cell-containing structure.

In some example implementations of the method, the perfusion liquid is delivered to both the second microfluidic channel and the third microfluidic channel to perform perfusion of the cell-containing structure from both lateral and vertical directions, thereby facilitating diffusion and/or advection between the first microfluidic channel and the second microfluidic channel, and facilitating diffusion and/or advection between the first microfluidic channel and the third microfluidic channel.

In some example implementations, the method further includes collecting the perfusion liquid after having contacted the perfusion liquid with the cell-containing structure, thereby obtaining collected perfusion liquid; and
detecting at least one secreted factor in the collected perfusion liquid, the secreted factor having been secreted by the viable cells within the cell-containing structure during perfusion.

In some example implementations of the method, the perfusion liquid is delivered to one of the second microfluidic channel and the third microfluidic channel, and wherein the other of the second microfluidic channel and the third microfluidic channel is employed to collect one or more secreted factors secreted by the viable cells within the cell-containing structure. The method may further include detecting at least one of the one or more secreted factors during perfusion of the cell-containing structure.

In some example implementations of the method, the cell-containing structure is formed by:
injecting a precursor liquid into the first microfluidic channel, the precursor liquid comprising the viable cells, the precursor liquid being confined within the first microfluidic channel by the lateral geometrical meniscus-pinning feature and the vertical geometrical meniscus-pinning feature; and
hardening the precursor liquid to form the cell-containing structure within the first microfluidic channel.

In some example implementations of the method, the cell-containing structure is formed by:
injecting a precursor liquid into the first microfluidic channel, the precursor liquid being confined within the first microfluidic channel by the lateral geometrical meniscus-pinning feature and the vertical geometrical meniscus-pinning feature; and
hardening the precursor liquid to form a gel structure within the first microfluidic channel;
injecting a cell-containing liquid into at least one of the second microfluidic channel and the third microfluidic channel, the cell-containing liquid comprising the viable cells; and
incubating the microfluidic device to facilitate perfusion of the gel structure with the viable cells, thereby forming the cell-containing structure.

In some example implementations, the method further includes imaging the cell-containing structure. The substrate may include a planar surface residing below the first horizontal planar region, such that the planar surface and the second horizontal planar region reside on opposite sides of the horizontal planar region, and wherein a portion of the substrate residing between the planar surface and the first microfluidic channel is substantially transparent, the method further comprising: imaging the cell-containing structure through the planar surface. The imaging may be performed during perfusion of the cell-containing structure.

In another aspect, there is provided a method of performing microfluidic perfusion of cell-containing structures, the method comprising:
providing a microfluidic device as described above;
forming cell-containing structures within the second microfluidic channel and the third microfluidic channel such that the cell-containing structures contain viable cells; and
injecting a perfusion liquid into the first microfluidic channel, the perfusion liquid comprising growth media; and
incubating the microfluidic device while contacting the perfusion liquid with the cell-containing structures.

In some example implementations, the method further includes:
collecting the perfusion liquid after having contacted the perfusion liquid with the cell-containing structure, thereby obtaining collected perfusion liquid; and
detecting at least one secreted factor in the collected perfusion liquid, the secreted factor having been secreted by the viable cells within the cell-containing structure during perfusion.

In some example implementations, the method further includes detecting at least one of the one or more secreted factors during perfusion of the cell-containing structure.

In some example implementations of the method, the cell-containing structures are formed by:
injecting a precursor liquid into the second microfluidic channel and the third microfluidic channel, the precursor liquid comprising the viable cells, the precursor liquid being confined within the second microfluidic channel by the lateral geometrical meniscus-pinning feature, and the precursor liquid being confined within the third microfluidic channel by the vertical geometrical meniscus-pinning feature; and
hardening the precursor liquid to form the cell-containing structure within the second microfluidic channel and the third microfluidic channel.

In some example implementations of the method, the cell-containing structures are formed by:
injecting a precursor liquid into the second microfluidic channel and the third microfluidic channel, the precursor liquid being confined within the second microfluidic channel by the lateral geometrical meniscus-pinning feature, and the precursor liquid being confined within the third microfluidic channel by the vertical geometrical meniscus-pinning feature; and
hardening the precursor liquid to form a gel structure within the second and third microfluidic channels;
injecting a cell-containing liquid into the first microfluidic, the cell-containing liquid comprising the viable cells; and
incubating the microfluidic device to facilitate perfusion of the gel structure with the viable cells, thereby forming the cell-containing structure.

In some example implementations, the method further includes imaging the cell-containing structure. The imaging may be performed during perfusion of the cell-containing structure.

A further understanding of the functional and advantageous aspects of the disclosure can be realized by reference to the following detailed description and drawings.

### BRIEF DESCRIPTION OF THE DRAWINGS

Embodiments will now be described, by way of example only, with reference to the drawings, in which:

FIGS. 1A, 1B and 1C illustrate an example multiplanar microfluidic device having laterally and vertically adjacent perfusion microfluidic channels.

FIGS. 2A and 2B show: (A) a multiplanar microfluidic system with polymerized hydrogel in the lower middle channel (MC) and water containing food coloring added to peripheral channels. Scalebar=8 mm. (B) An upper channel plane is situated directly above the lower channel plane to provide vertically accessible top channels, thus presenting an opportunity for lateral and normal (vertical) diffusion to take place.

FIG. 2C illustrates an example method of loading the central microfluidic channel and adjacent perfusion microfluidic channels of an example multiplanar microfluidic device.

FIG. 2D illustrates an example multiplanar microfluidic device having detachably removable layers that optionally facilitate access to a gel structure.

FIG. 2E illustrates an example multiplanar microfluidic device having an additional reservoir configured to provide indirect fluidic access to the vertically-offset microfluidic channel through a membrane.

FIG. 2F illustrates an example four-step loading process for forming a gel structure with multiple cell types within the central microfluidic channel of a multiplanar microfluidic device. The hydrogel component can first be loaded into the central microfluidic channel and allowed to polymerize while pinned by an elongate meniscus-pinning edge features. Each of the other three channels can then be used for seeding various different cell types and supplying media.

FIGS. 3A, 3B, 3C and 3D show: (A) Channel geometries micromachined in individual device layers using a CNC milling machine that executes G-code instructions from 3D CAD models. These layers are assembled to produce multiplanar channel geometries with an elongate meniscus-pinning edge features arranged orthogonally in vertical and horizontal pairs. (B) (Left) Photographs of the micromilled and bonded layers resulting in a complete multiplanar microfluidic chip with four systems. (Right) Locations of retention grooves in relation to fluid volumes and device edges. (C) Parity plot indicating precision and accuracy of machined phase guide pairs over a range of heights (25, 50, 75, 100, 125, 150, 175 and 200 μm) at two different widths (100 and 200 μm). (D) 2× fluorescence images of hydrogel pinning for each phase guide height/width combination. Devices were tested with 2.5 mg/mL type I collagen containing ˜1.6% 1-μm green fluorescent microbeads.

FIGS. 4A, 4B, 4C and 4D illustrate alternative example multiplanar microfluidic configurations. (A) Center-load configuration with total of 4 channels. (B) Side-load configuration with total of 5 channels. (C) Expanded center-load configuration with total of 7 channels. (D) Expanded side-load configuration with total of 8 channels. Each subfigure shows: isometric view of CAD model (top); cross-section (black icon); image of 1-μm fluorescent microbeads pinned by phase guides (bottom left); photograph of complete chip containing 4 systems (bottom middle); and top-down brightfield image of the fabricated channel arrangement. Fluorescence images taken at 2×. Scalebars=2 mm.

FIGS. 5A, 5B and 5C illustrate example expanded multi-device configurations implemented on fluidic chips.

FIGS. 6A and 6B illustrate example expanded multi-device configurations implemented in microplate formats.

FIGS. 7 and 8 illustrate alternative inverted configurations for loading multiplanar microfluidic devices.

FIGS. 9A, 9B, 9C, 9D and 9E demonstrate validation of the computational transport model using two-channel geometry. (A) Illustration of the diffusion model setup based on the two-channel fabricated geometry, with 10-kDa FITC-Dextran allowed to diffuse over 3 h from left channel to right channel; right channel contains 2.5 mg/mL type I collagen. (B) Surface plot of the sum of squared residuals (SSR) for simulation curves generated based on a range of porosity and diffusivity values. The plot was then used to obtain fitted parameter values for the no-cell (control) condition. (C) SSR over a range of monolayer diffusivity values which were used to find the fitted parameter value for the with-cell condition. (D) Measured (meas.) and simulated results (sim.) for the normalized change in concentration over the 3-h period for both the control (− cells) and with-cell (+ cells) conditions. (E) Timelapse sequences for the first hour of the experiment where 10-kDa FITC-Dextran diffused from the left channel to the right channel containing the hydrogel (upper row—control condition with no cells; lower row—with cell monolayer between channels).

FIG. 10 is a table providing the parameter values used in diffusion simulations.

FIGS. 11A, 11B, 11C, 11D, 11E, 11F, 11G and 11H provide simulations of single-plane (SP) and dual-plane (DP) designs. (A) Illustration of cross-sections of SP and DP designs and direction of diffusion from peripheral channels into the hydrogel, with barrier resistance from cell monolayers in the side channels. (B and F) Simulated spatial distribution of concentration in DP and SP designs over 3 h. (C and G) Normalized concentration over time in the hydrogel for DP and SP designs. (D and H) Normalized concentration over time in DP configuration comparing top to side channels. (E) Illustration of cross-sections of SP and DP designs and direction of diffusion from the hydrogel outward to peripheral channels, with barrier resistance from cell monolayers in the side channels.

FIGS. 12A, 12B, 12C, 12D and 12E show results from cell culture and viability of human umbilical vein endothelial cells (HUVECs) and pancreatic stellate cells (FSCs). (A) Fluorescence images showing viability of PSCs embedded in a type I collagen gel at 24 h and 72 h in both SP and DP designs, and with or without HUVEC monolayers. Images shown are maximum intensity z projections taken at 10× magnification. Scalebars=250 μm. (B) Top view confocal image of the left (LC), middle (MC) and right channels (RC), with the middle containing collagen mixed with 1-μm green-fluorescent microbeads. Scalebar=250 μm. (C) Confocal image of cross-sectional view of left and middle channels with the collagen pinned by the elongate meniscus-pinning edges feature and forming a curved interface. Scalebar=200 μm. (D) Confocal image of cell monolayer along curved gel surface. MB=microbeads in gel; dashed ellipses indicate regions with VE-cadherin on HUVECs; solid ellipses indicate regions with Hoechst nuclear stain. Scalebar=250 μm. (E) Endothelial monolayer with VE-cadherin localization at cell borders. Scalebar=150 μm.

FIG. 13 schematically illustrates the design iteration progression. Three of the most notable iterations are shown. Initial designs involved relatively long port necks and phase guide lengths with sharp cornering and a lack of reservoirs. Intermediate designs improved gel pinning and reduced bubble nucleation through the use of shortened phase guide lengths and curved geometries. Reservoirs were added to provide sufficient nutrient supply to cultures over a 24-h period. The current design builds upon these modifications by adding an upper channel system that is vertically accessible by the central channel to improve nutrient access to cells embedded in the gel. The lowest row of images shows the result of collagen polymerization within the device, visualized with green fluorescent micro beads.

FIGS. 14A, 14B, 14C, 14D, 14E and 14F demonstrates the significance of channel capacity in simulation models. (A) and (B) show the rates of species diffusion in a two-channel system over a 24-h time period for models including and omitting the channel geometries respectively. Similarly, (C), (D) and (E), (F) pairs show results for the three-channel (NTC) system and four-channel (TC) system respectively.

FIGS. 15A, 15B, 15C and 15D illustrate an example multiplanar microfluidic device in which flow is restricted between adjacent microfluidic channels via meniscus-pinning surface regions having local hydrophilic and hydrophobic regions.

FIGS. 16A, 16B, 16C and 16D show results from experiments involving reflected light confocal imaging of collagen fibers in situ.

FIGS. 17A, 17B and 17C show results from experiments designed to investigate on-chip stiffness using Atomic Force Microscopy (AFM).

### DETAILED DESCRIPTION

Various embodiments and aspects of the disclosure will be described with reference to details discussed below. The following description and drawings are illustrative of the disclosure and are not to be construed as limiting the disclosure. Numerous specific details are described to provide a thorough understanding of various embodiments of the present disclosure. However, in certain instances, well-known or conventional details are not described in order to provide a concise discussion of embodiments of the present disclosure.

As used herein, the terms “comprises” and “comprising” are to be construed as being inclusive and open ended, and not exclusive. Specifically, when used in the specification and claims, the terms “comprises” and “comprising” and variations thereof mean the specified features, steps or components are included. These terms are not to be interpreted to exclude the presence of other features, steps or components.

As used herein, the term “exemplary” means “serving as an example, instance, or illustration,” and should not be construed as preferred or advantageous over other configurations disclosed herein.

As used herein, the terms “about” and “approximately” are meant to cover variations that may exist in the upper and lower limits of the ranges of values, such as variations in properties, parameters, and dimensions. Unless otherwise specified, the terms “about” and “approximately” mean plus or minus 25 percent or less.

It is to be understood that unless otherwise specified, any specified range or group is as a shorthand way of referring to each and every member of a range or group individually, as well as each and every possible sub-range or sub-group encompassed therein and similarly with respect to any sub-ranges or sub-groups therein. Unless otherwise specified, the present disclosure relates to and explicitly incorporates each and every specific member and combination of sub-ranges or sub-groups.

As used herein, the term “on the order of”, when used in conjunction with a quantity or parameter, refers to a range spanning approximately one tenth to ten times the stated quantity or parameter.

Unless defined otherwise, all technical and scientific terms used herein are intended to have the same meaning as commonly understood to one of ordinary skill in the art. Unless otherwise indicated, such as through context, as used herein, the following terms are intended to have the following meanings:

As used herein, the phrase “geometrical meniscus-pinning feature” refers to a geometrical feature, such as a protrusion or recess or a sharp corner, that resides between adjacent microfluidic channels, in the absence of a membrane, and is capable of forming a capillary pressure or interfacial barrier suitable for prohibiting transverse fluid flow from one microchannel to another microchannel, as a consequence of meniscus pinning by the geometrical feature. Non-limiting examples of geometrical meniscus-pinning features include an elongate meniscus-pinning edge feature extending longitudinally along a contact region between adjacent microfluidic channels (e.g. a “phaseguide”) and an array of microposts or other meniscus pinning structures that extend along the contact region between adjacent microfluidic channels.

As used herein, the phrase “direct fluid communication” refers to fluidic communication between one microfluidic channel and another microfluidic channel in the absence of an intervening membrane.

As described above, biomicrofluidic systems offer unique advantages over traditional cell culture devices in their ability to provide precise spatiotemporal control and create complex microenvironments via the controlled application of multiple physical, biochemical and mechanical cues. These advantages are facilitated often by device implementations in which adjacent microfluidic channels are brought into direct or indirect fluid communication, thereby permitting controlled interactions between fluids and/or tissue structures residing in the adjacent microfluidic channels.

Unfortunately, existing biomicrofluidic devices are limited in their ability to reproduce the complex, three-dimensional environments that exist in real biological systems due to spatial constraints placed on interactions between adjacent microfluidic channels. Indeed, most of the biomicrofluidic devices that have been implemented to date have been limited to laterally-adjacent microfluidic channel interactions in a single horizontal plane, or to vertically-stacked single-channel arrangements. As a result, lateral and vertical (or normal/out-of-plane) diffusion have been isolated to their respective designs only, and such devices have been unable to mimic the delivery of nutrients and 3D communication that typifies in vivo microenvironments.

Furthermore, existing devices that combine both lateral and vertical fluidic communication between adjacent microfluidic channels have been limited to implementations that restrict vertical fluidic interactions via the use of an intervening membrane, which the present inventors have found can present significant disadvantages when seeking to replicate a complex three-dimensional cell culturing environment. Indeed, the need for a membrane to provide a physical barrier between vertically-adjacent microfluidic channels prevents direct fluidic communication between vertically-adjacent microfluidic channels. This lack of direct fluidic communication in the vertical direction can lead to inefficient diffusion in the vertical direction. In addition, the intervening membrane in such devices is commonly a polymeric material that does not accurately mimic the matrix composition and biochemistry of native basement membranes. Moreover, such designs can be problematic in that they can result in a microenvironment in which the rate of diffusion among adjacent microfluidic channels differs significantly in the horizontal direction as compared to the vertical direction, thereby introducing artificial anisotropies in the local cell culture environment. Furthermore, the assembly of a fully bonded membrane feature in such devices also significantly reduces the ease of fabrication and assembly, thereby restricting the scalability of manufacture and the potential for high volume production.

The present disclosure provides various example biomicrofluidic systems and devices that overcome the aforementioned limitations and problems by employing multiplanar configurations that facilitate direct fluid communication of a microfluidic channel with laterally and vertically adjacent microfluidic channels. Such devices may be employed to facilitate both lateral and vertical (normal) diffusion to a microfluidic channel from adjacent microfluidic channels, without requiring the presence of an intervening membrane, thereby achieving improved fluid communication and associated diffusion.

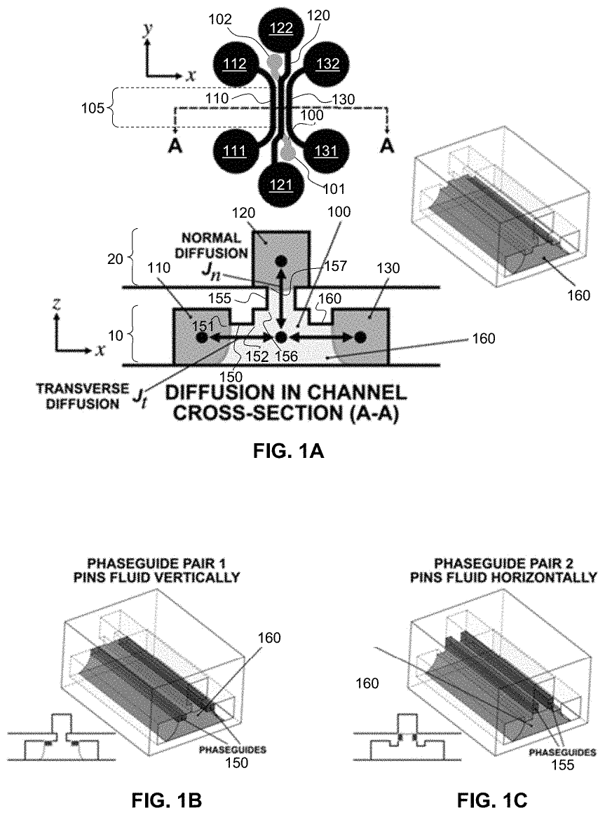

With reference to FIG. 1A, an example multiplanar microfluidic device embodiment is shown that includes a first microfluidic channel 100 and a second microfluidic channel 110, both of which are defined within a first horizontal planar region 10. The first microfluidic channel 100 resides laterally adjacent to the second microfluidic channel 110 along an elongate portion of 105 of the first microfluidic channel and is brought into direct lateral fluid communication with the second microfluidic channel 110 within this portion, as shown in the cross-sectional view in the lower portion of the figure.

The device also includes a third microfluidic channel 120 residing within a second horizontal planar region 20 that is vertically offset from the first planar region 10. The first microfluidic channel 100 resides vertically adjacent to the third microfluidic channel 120 over the elongate fluid communication portion 105 of the first microfluidic channel and is brought into direct vertical fluid communication with the second microfluidic channel 120, as shown in the cross-sectional view in the lower portion of the figure.

Both the second microfluidic channel 110 and the third microfluidic channel 120 are in direct fluid communication with the first microfluidic channel 100, without the presence of an intervening membrane.

As shown in FIGS. 1A-1C, a lateral geometrical meniscus-pinning feature 150 is provided between the first microfluidic channel 100 and the second microfluidic channel 110 within the elongate fluid communication portion of 105 the first microfluidic channel 100. When a fluid is delivered to one of the first microfluidic channel 100 and the second microfluidic channel 110, the feature 150 causes meniscus pinning (forming a capillary pressure barrier) that resists lateral fluid flow between the first microfluidic channel 100 and the second microfluidic channel 110, thereby facilitating the loading of one of the first microfluidic channel 100 or the second microfluidic channel 110 with a fluid while preventing the fluid from entering the other of the first microfluidic channel 100 and the second microfluidic channel 110.

Likewise, a vertical geometrical meniscus-pinning feature 155 resides between the first microfluidic channel 100 and the third microfluidic channel 120 within the elongate fluid communication portion 105 of the first microfluidic channel 100. The vertical geometrical meniscus-pinning feature 155 resists fluid flow between the first microfluidic channel 100 and the third microfluidic channel 120 when a fluid is delivered to one of the first microfluidic channel 100 and the third microfluidic channel 120, thereby facilitating the loading of one of the first microfluidic channel 100 or the third microfluidic channel 120 with a fluid while preventing the fluid from entering the other of the first microfluidic channel 100 and the third microfluidic channel 120.

The example device shown in FIGS. 1A-1C employs a non-limiting example type of geometrical meniscus-pinning feature, for both the lateral and vertical features 150 and 155, in the form of an elongate meniscus-pinning edge feature (e.g. a “phaseguide”) that extends longitudinally between the first microfluidic channel and the second microfluidic channel over the elongate fluid communication portion 105. As shown in the figures, the lateral geometrical meniscus-pinning feature 150 is provided by a protrusion that forms first and second longitudinal edges 151, 152 that extend longitudinally over the elongate fluid communication portion 105. Likewise, the vertical geometrical meniscus-pinning feature 155 is provided by a protrusion that forms first and second longitudinal edges 156, 157 that extend longitudinally over the elongate fluid communication portion 105. While each example geometrical meniscus-pinning edge feature includes two longitudinal edges, other example implementations may employ a single longitudinal edge (e.g. a single step in channel width or thickness).

It will be understood that other types of geometrical meniscus-pinning features may be employed in the alternative. For example, while the example geometrical meniscus-pinning ridge features are defined by protrusions, one or more geometrical meniscus-pinning ridge features may instead be defined by a recess, or, for example, a combination of a recess and a protrusion.

In other example implementations, other types of geometrical meniscus-pinning features may be employed. In general, such meniscus pinning features are absent of membrane structure and employ a capillary barrier structure to energetically disfavour advance of the meniscus beyond the meniscus pinning feature. One example of another type of meniscus pinning feature is an array of meniscus pinning elements, such as, but not limited to, an array or microposts.

Moreover, while the example device shown in FIGS. 1A-1C employs a common type of geometrical meniscus-pinning feature for both the lateral geometrical meniscus-pinning feature and the vertical geometrical meniscus-pinning feature, it will be understood that different types of geometrical meniscus-pinning features may be employed for each geometrical meniscus-pinning feature. For example, the lateral geometrical meniscus-pinning feature may be defined by a micropost array and the vertical geometrical meniscus-pinning feature may be defined by a geometrical meniscus-pinning edge feature.

As shown in FIGS. 1A-1C, a fourth microfluidic channel 130 may optionally be provided within the first horizontal planar region 10, on an opposite side of the first microfluidic channel 100 relative to that of the second microfluidic channel 110, in order to facilitate fluid communication from a second horizontal direction, such that the first microfluidic channel 100 is contacted by microfluidic channels on both lateral sides and a vertical direction. As shown in the figure, the fourth microfluidic channel 130 resides laterally adjacent to the first microfluidic channel 100 along the elongate portion of 105 of the first microfluidic channel and is brought into direct lateral fluid communication with the first microfluidic channel 100 within this portion, as shown in the cross-sectional view in the lower portion of the figure. Furthermore, a second lateral geometrical meniscus-pinning feature 160 is provided between the first microfluidic channel 100 and the fourth microfluidic channel 130 within the elongate fluid communication portion of 105 the first microfluidic channel 100, such that when a fluid is delivered to one of the first microfluidic channel 100 and the fourth microfluidic channel 130, the feature 160 causes meniscus pinning that resists fluid flow between the first microfluidic channel 100 and the fourth microfluidic channel 130, thereby facilitating the loading of one of the first microfluidic channel 100 or the fourth microfluidic channel 130 with a fluid while preventing the fluid from entering the other of the first microfluidic channel 100 and the fourth microfluidic channel 130. While the inclusion of the second microfluidic channel 110 and the third microfluidic channel 120 facilitate bi-directional microfluid communication with the first microfluidic channel 100 from lateral and vertical directions, the optional inclusion of the fourth microfluidic channel 130 beneficially facilitates fluid communication with the first microfluidic 100 channel from both lateral directions.

The device configuration illustrated in FIG. 1A demonstrates a non-limiting example implementation in which the elongate fluid communication portion 105, over which the adjacent microfluidic channels are in direct fluid communication, and over which lateral channel-to-channel flow is resisted via the geometrical meniscus-pinning features, is common for the lateral pair of adjacent microfluidic channels (the first and second microfluidic channels) and the vertical pair of adjacent microfluidic channels (the first and third microfluidic channels). In other words, at every location along the first microfluidic channel for which direct lateral fluid communication exists via contact between the first and second microfluidic channels, direct vertical fluid communication also occurs via contact between the first and third microfluidic channels (and in the case of the optional inclusion of the fourth microfluidic channel, direct lateral fluid communication also occurs via contact between the first and fourth microfluidic channels).

However, in other example implementations, one or more pairs of adjacent microfluidic channels may have different elongate fluid communication portions 105 that partially overlap with one another, or in some cases, do not overlap with one another. For example, a first elongate fluid communication portion that defines the portion of the first microfluidic channel 100 along which direct fluid communication occurs between the first microfluidic channel 100 and the second microfluidic channel 110 may be different that a second elongate fluid communication portion that defines the portion of the first microfluidic channel 100 along which direct fluid communication occurs between the first microfluidic channel 100 and the third microfluidic channel 120.

Furthermore, although FIGS. 1A-1C illustrate an example case in which a single elongate fluid communication portion exists for each respective pair of adjacent microfluidic channels, in other example implementations, a given pair of adjacent microfluidic channels may be brought into contact at two or more different locations along the first microfluidic channel.

Referring again to FIG. 1A, the upper portion of the figure shows an overhead view that illustrates the example inclusion of ports and/or reservoirs connected to the ends of the microfluidic channels. The laterally adjacent microfluidic channels 110 and 130 are connected to respective reservoirs 111, 112 and 131, 132, and the vertically microfluidic channel 120 is connected to reservoirs 121 and 122. The central microfluidic channel 100 is connected to reservoirs/ports 101 and 102 that have a smaller volume that the reservoirs of the adjacent microfluidic channels, for example, in order to reduce the dead volume (the volume beyond the first microfluidic channel 100), and/or to permit direct engagement with a pipette tip. This configuration may be useful in applications for which the fluid delivered to the first microfluidic channel 100 is derived from a small sample volume.

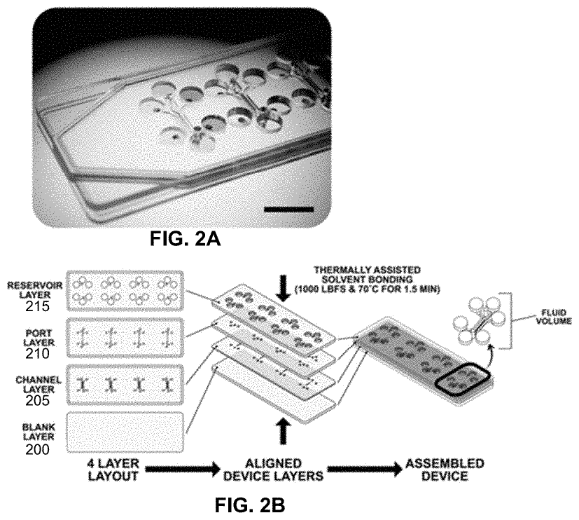

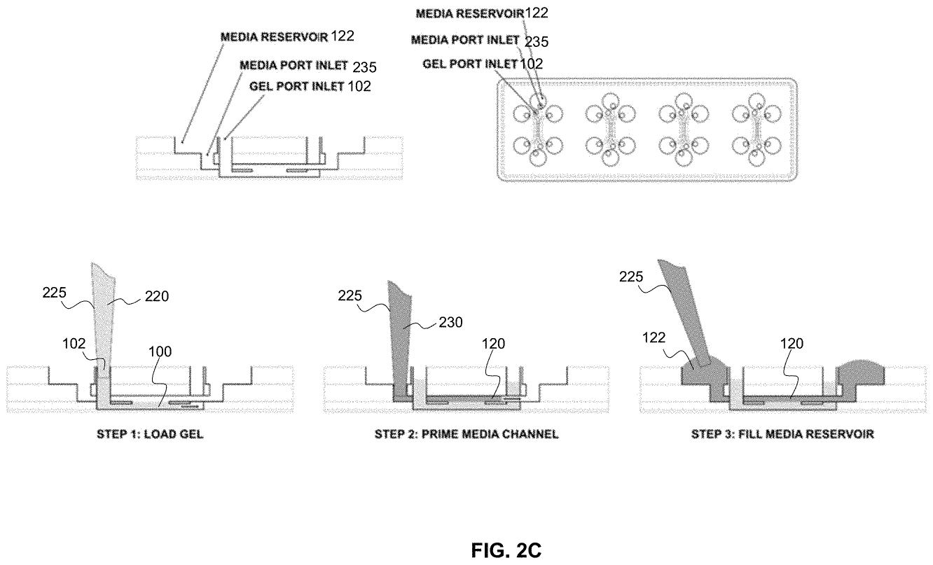

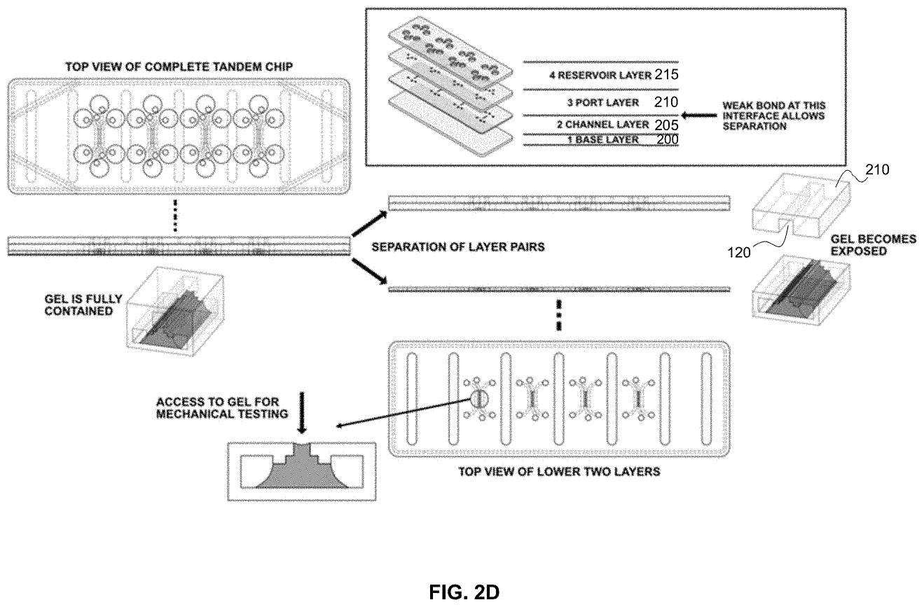

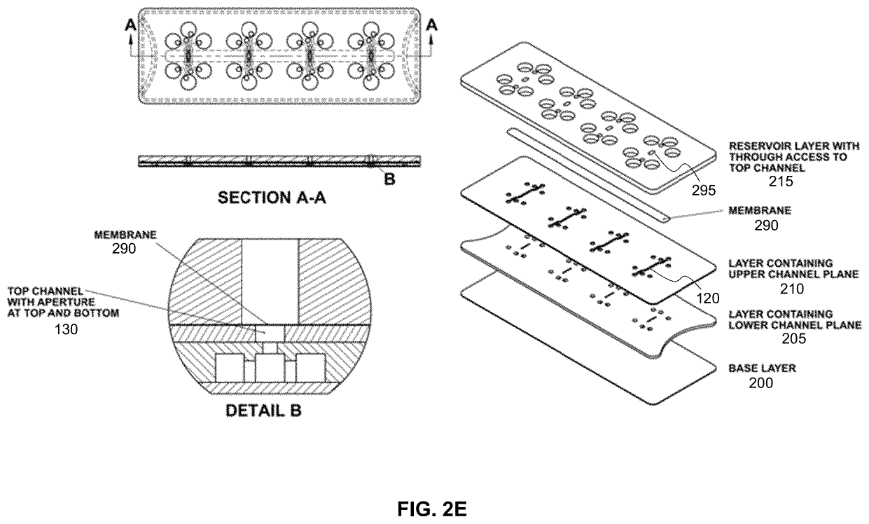

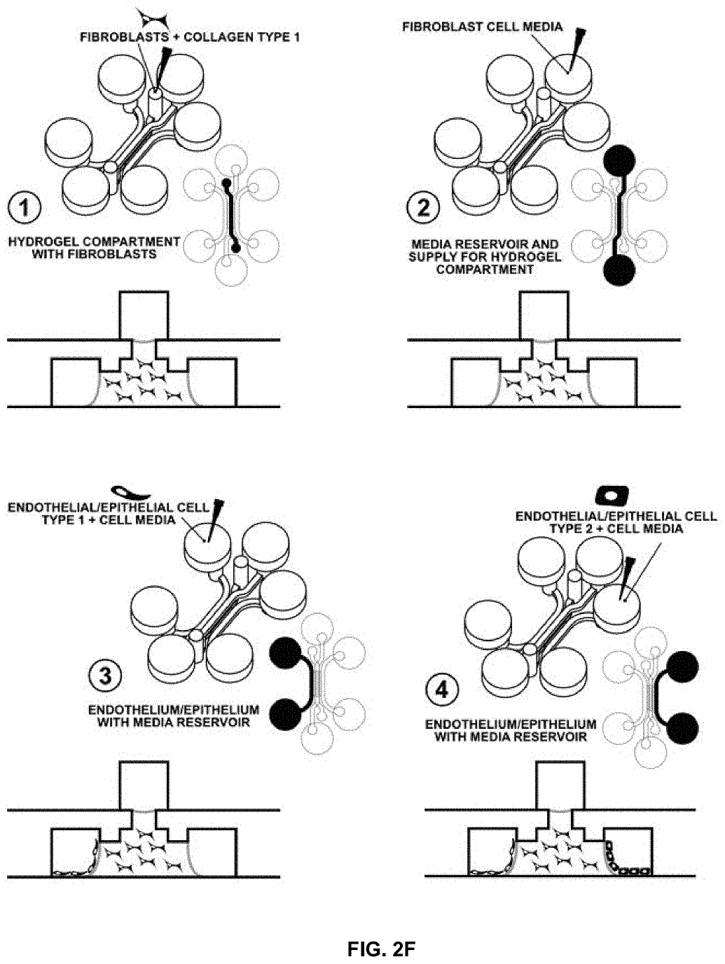

The present example multiplanar microfluidic devices, and variations and adaptations thereof, may be fabricated according to using a variety of materials and fabrication methods. In some example implementations, a multiplanar microfluidic device with integrated lateral and vertical meniscus pinning features may be fabricated as a stacked set of layers, as shown, for example, in FIGS. 2A and 2B, where FIG. 2A is a photograph of a fully assembled multilayer device and FIG. 2B shows the stacking of the multiple layers to form the device.

The example multilayer device and assembly method illustrated in FIG. 2B employs four example device layers to fabricate an example multi-device fluidic chip. The multi-device fluidic chip includes four multiplanar microfluidic devices, with each device including a first (central) microfluidic channel, second and fourth microfluidic channels that are laterally adjacent to and in direct lateral fluid communication with the first microfluidic channel, and a third microfluidic channel that is vertically adjacent to and in direct vertical fluid communication with the first microfluidic channel. The example device layers include (i) a blank layer 200 (base substrate), (ii) first horizontal planar layer 205 that includes the first microfluidic channel and the laterally adjacent second and fourth microfluidic channels, (ii) a second horizontal planar layer 210 that includes the third microfluidic channel that is vertically adjacent from the first microfluidic channel; and through-ports for accessing the first, second and fourth microfluidic channels, and (iv) an upper reservoir layer 215 that includes reservoirs in fluid communication with the second, third and fourth microfluidic channels; and ports in fluid communication with the first microfluidic channel.

In the example configuration illustrated in FIGS. 2A and 2B, a reservoir top layer 215 is provided for use in static culture conditions. However, it will be understood that this configuration is but one example implementation and that other device configurations may be employed in the absence of reservoirs. For example, the present example reservoir layer 215 can be replaced with a port connection layer to facilitate world-to-chip connections, for example, when shear flow and media perfusion are desirable.

FIG. 2C illustrates an example method of loading the central microfluidic channel of an example multiplanar microfluidic device to form a gel structure and filling the laterally and vertically adjacent perfusion microfluidic channels with a perfusion liquid that includes culture media. As shown FIG. 2C, a gel precursor 220 is first loaded into the central microfluidic channel 100 using a pipette 225 by contacting the distal end of the pipette tip with a port 102 of the central microfluidic channel 100. After the gel has been formed, at least one of the adjacent perfusion microfluidic channels (the figure shows vertically adjacent microfluidic channel 130) is primed with the perfusion liquid 230 using the pipette via contact of the pipette 225 with an inlet (port) 235 of the adjacent perfusion microfluidic channel 130. The media (perfusion) reservoir 122 that is in fluid communication with the primed perfusion microfluidic channel 120 is then filled with the perfusion liquid 230.

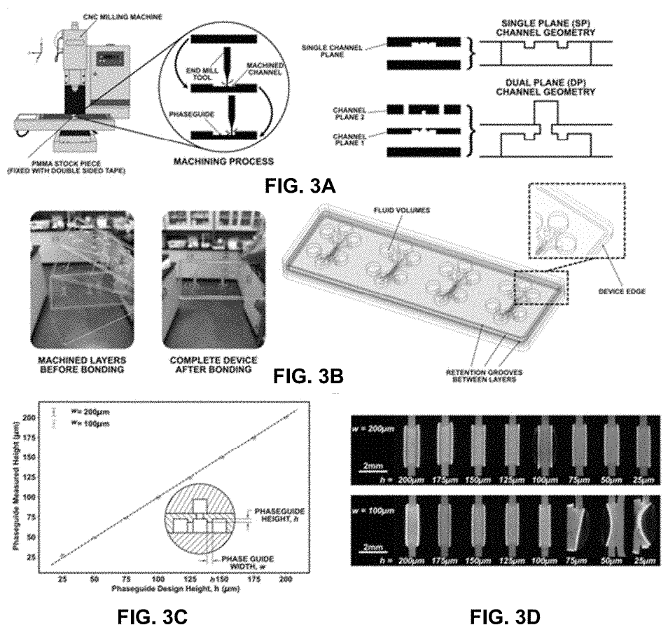

The geometrical meniscus-pinning features may be defined, for example, in the second device layer, for example, as illustrated in FIGS. 3A and 3B. For example, as illustrated, the upper surface of the first layer 200 or a lower surface of the second layer 205 may include recessed features that define, after the first layer 200 is bonded to the second layer 205, the first microfluidic channel, the second microfluidic channel, and the lateral geometrical meniscus-pinning feature. The second layer 205 may also include an aperture defined such that the aperture resides above the first microfluidic channel after the first layer is bonded to the second layer. A third layer 210 may be secured to the second layer 205, where a lower surface of the third layer 210 includes an additional recessed feature that defines, after the third layer is bonded to the second layer, the third microfluidic channel 120, such that the aperture forms, at least in part, the vertical geometrical meniscus-pinning feature.

Non-limiting examples of suitable materials for forming the device layers include thermoplastics such as poly(methyl methacrylate) (PMMA), polystyrene (PS), polycarbonate (PC) and the cyclic olefin polymer family (COC, COP, CBC), elastomers such as polydimethylsiloxane (PDMS), styrenic elastomeric polymers, polypropylene (PP), transparent acrylonitrile butadiene styrene (ABS), polytetrafluoroethylene (PTFE), and glass.

In the present example devices, PMMA was selected as a device material to avoid problems associated with PDMS and facilitate manufacturing at scale. Additionally, it was found that PMMA is attractive due to its ease of machining, cleaning and bonding, its optical clarity, and its relative bioinertness compared to other commonly used materials.

Non-limiting examples of fabrication methods for forming microfluidic channel features and geometrical meniscus-pinning features in polymer-based microfluidic device layers include micromilling, hot embossing and injection molding of thermoplastics, soft lithography of PDMS, and chemical etching and laser ablation of silicon and glass substrates. For example, thermoplastic micromilling may be employed for device fabrication as it allows rapid iteration and conversion from computer-aided design (CAD) models to finished device and can be scaled to medium volume production when integrated with other processes such as hot embossing.

Fabricated device layers may be aligned, stacked and bonded/laminated using various examples methods, such as, but not limited to, thermally assisted solvent bonding, surface activation via oxygen plasma, ultrasonic welding, epoxy and tape adhesives, anodic bonding for silicon and glass substrates, and mechanical clamping and fastening.

In some example implementations, the device layer that, upon bonding to an underlying layer, encloses the first microfluidic channel, may be removably attached to the underlaying layer, in order to permit detachment and access to a structure, such as a cell-containing gel structure, residing within the first microfluidic channel, for example for subsequent analysis. Such an example implementation is illustrated in FIG. 2D.

For example, in the case of a gel that is formed in the first microfluidic channel, after disassembly of the layers, the top of the gel that resides in the first microfluidic channel is exposed to the atmosphere, unimpeded by the third microfluidic channel that was vertically positioned above it. In particular, if the third microfluidic channel were absent, and the gel that resides in the first microfluidic channel was instead in contact with the surface above it, the gel would be attached to that surface, and any disassembly of the layers would lead to disruption or distortion of the gel, rendering it unacceptable as an unbiased experimental sample. The exposed gel may be subjected to measurements such as, for example, microscopy and stiffness measurement such as with atomic force microscopy. The exposed gel may additionally or alternatively be subject to further assaying and analysis, such as, for example, paraffin embedding, histological sectioning and staining. In other example implementations, the exposed gel may be measured for matrix loss, the gel may be digested to isolate the cells (e.g., for single-cell RNA sequencing, flow cytometry, etc.) and the gel surface may be imaged using scanning electron microscopy.

FIG. 2E illustrates an example implementation in which the vertically-adjacent perfusion microfluidic channel 120 is indirectly brought into fluid communication with an upper reservoir 290 through a horizontal membrane 295. This implementation allows, as a non-limiting example, ex vivo tissue explants or other living biological cells to be deposited and cultured directly in the upper reservoir 200 to then interact with the vertically-adjacent perfusion microfluidic channel 120 by diffusion.

As explained above, the incorporation of the geometrical meniscus-pinning features between the adjacent microfluidic channels provides a capillary barrier that resists flow from a fluid within one of the adjacent microfluidic channels to the other of the adjacent microfluidic channels.

The dimensions of the geometrical meniscus-pinning features, such as elongate meniscus-pinning edge features, may be experimentally selected and/or optimized by maximizing fluid pinning capability while factoring the constraints of micromilling. For example, the heights and widths of elongate meniscus-pinning edge feature structures may be minimized to maximize the cross-sectional area for diffusive flux, while ensuring that the heights are not too small in comparison to overall channel heights such that they fail to establish sufficient interfacial tension at the surface and lead to fluid overflow into adjacent microfluidic channels. Furthermore, thinner edge features may be selected to bring adjacent channels closer together, but with a thickness selected to provide sufficient structural stiffness to avoid fracture during fabrication (e.g. due the cyclic loading of the micromilling tool during the milling process).

The dimensions of the geometrical meniscus-pinning features may be selected and/or optimized for use with specific fluid types, as different fluids may exhibit varying surface tension interactions that warrant different dimensional properties to stabilize meniscus pinning and maximize the diffusive interaction area between adjacent microfluidic channels. For example, channel and/or geometrical meniscus-pinning features may be empirically optimized over multiple iterations to improve hydrogel pinning quality and repeatability.

Referring again to FIG. 1A, lateral microfluidic channels that gradually curve inward to contact the first microfluidic channel may be beneficial in reducing bubble nucleation that can otherwise occur in the presence of sharp corners in the channels. Furthermore, the present inventors observed that pinning repeatability of hydrogels within the first microfluidic channels improved as the length of the elongate fluid communication portion 105 was reduced.

The capillary barrier provided by the geometrical meniscus-pinning feature may be employed to selectively fill one of the microfluidic channels without filling one or more of the remaining adjacent microfluidic channels. For example, as shown in FIGS. 1A-1C, the first microfluidic channel 100 may be filled with a gel precursor liquid (e.g. a hydrogel precursor liquid) that is retained within the first microfluidic channel 100 by the geometrical meniscus-pinning features. The gel precursor may then be hardened (solidified) to form a gel 160 within the first microfluidic channel 100.

The present example multiplanar microfluidic devices may be employed in a wide variety of applications. For example, a cell-containing structure may be formed within the first microfluidic channel such that the cell-containing structure contains viable cells. A perfusion liquid containing culture media may then be delivered into one or both of the second microfluidic channel and the third microfluidic channel, such that the perfusion media comes into direct fluid communication with the cell-containing structure, thereby facilitating the delivery of the culture media, via diffusion and/or advection, to the cell-containing structure via both lateral and vertical directions. The device may be incubated while contacting the perfusion liquid with the cell-containing structure to maintain cell viability, and/or to facilitate cell proliferation and/or differentiation.

In one example implementation, the perfusion liquid may be collected after having contacted the perfusion liquid with the cell-containing structure. In another example implementation, the perfusion liquid may be delivered to one of the adjacent microfluidic channels (e.g. the second microfluidic channel or the third microfluidic channel), and another one of the adjacent microfluidic channels may be employed to collect one or more secreted factors secreted by the viable cells within the cell-containing structure. The collected perfusion liquid may be analyzed to detecting a secreted factor that was secreted by the viable cells within the cell-containing structure during perfusion.

As noted above, the cell-containing structure may be a gel that is formed by injecting a precursor liquid into the first microfluidic channel, where the precursor liquid includes viable cells, such that the precursor liquid is confined within the first microfluidic channel by the lateral geometrical meniscus-pinning feature and the vertical geometrical meniscus-pinning feature. The cell-containing precursor liquid residing within the first microfluidic channel may be hardened to form the cell-containing structure within the first microfluidic channel. Alternatively, the cell-containing structure may be formed by injecting a precursor liquid into the first microfluidic channel such that the precursor liquid is confined within the first microfluidic channel by the lateral geometrical meniscus-pinning feature and the vertical geometrical meniscus-pinning feature. The precursor liquid may be hardened to form a gel structure within the first microfluidic channel. A cell-containing liquid may be injected into at least one of the adjacent microfluidic channels (e.g. the second microfluidic channel or the third microfluidic channel) and the microfluidic device may be incubated to facilitate diffusive transport cells from the cell-containing liquid to the gel structure, thereby forming the cell-containing structure.

In some example implementations, a cell-containing perfusion liquid may be delivered to the second and/or third microfluidic channel after having formed a gel within the first microfluidic channel. These cells, delivered by the cell-containing perfusion liquid, can then settle on the gel interface of the gel structure and attach to form cell sheets or monolayers.

In some example implementations, viable cells within the cell-containing structure in the first microfluidic channel may secrete extracellular matrix proteins that are then incorporated into the existing gel, leading to matrix remodeling. The viable cells within the cell-containing structure in the first microfluidic channel may secrete matrix degradation proteins (e.g., matrix metalloproteinases) that can break down or degrade some of the existing gel structure, leading to matrix remodeling. Furthermore, the matrix remodeling may be manifested as a change in the gel stiffness, where the stiffness of the gel may increase when new matrix proteins are deposited, or may decrease when matrix metalloproteinases degrade the gel.

In some example implementations, a second cell type may be injected into the second microfluidic channel, and allowed to settle onto the gel interface created by the position of the gel within the first microchannel due to pinning of the gel with the lateral geometrical meniscus-pinning feature. This second cell type may form a layer of cells, such as, for example, a confluent monolayer (of endothelial or epithelial cells) to serve as a cell barrier. A third cell type may be injected into the third microfluidic channel and allowed to settle onto the gel interface created, and this third cell type may also form a layer of cells, such as a confluent monolayer (of endothelial or epithelial cells) to serve as a cell barrier. Furthermore, a fourth cell type may be injected into the second microfluidic channel, and allowed to settle onto the cell barrier created by the second cell type, and this fourth cell type can then transmigrate through the cell barrier and enter the gel. Any of the cell types may migrate or “invade” inside the gel, and as described below, the migration may be imaged through the bottom substrate using such microscopy techniques such as confocal microscopy. An example method for the formation of a gel structure and the incorporation of multiple cell types into and/or onto the gel structure is illustrated in FIG. 2F.

In some example implementations, one or more microfluidic channels of the multiplanar device may be imaged, for example, using a microscope. For example, a cell-containing structure formed within the first microfluidic channel and cultured via perfusion of one or more of the adjacent microfluidic channels may be imaged through the bottom surface of the device. In such a case, the multiplanar microfluidic device may include a substrate having a planar surface residing below the first horizontal planar region, where at least the portion of the substrate residing between the planar surface and the first microfluidic channel is substantially transparent.

Imaging of the cell-containing structure may be employed for a wide variety of uses and applications. For example, images may be obtained of viable cells that were initially delivered together with the gel precursor into the first microfluidic channel. Images may additionally or alternatively be obtained of a second cell type, some of which may migrate from a cell-containing perfusion liquid provided within the second microfluidic channel into the cell-containing structure within the first microfluidic channel, and/or a second cell type, some of which may migrate from another cell-containing perfusion liquid provided within the third microfluidic channel into the cell-containing structure within the first microfluidic channel, and/or a fourth cell type, some of which may migrate into the cell-containing structure by transmigrating through the cell barrier created by second or third cell type.

The example embodiments disclosed herein significantly improve over previously reported devices due to inclusion of one or more laterally-adjacent perfusable microfluidic channels that come into direct fluid communication with the first microfluidic channel within same plane (lateral direction) as the first microfluidic channel, as well as one or more vertically vertically-adjacent, out-of-plane perfusable microfluidic channels “stacked” above the first plane (normal direction), thus creating multidirectional connectivity between microchannels. Diffusion and advection transport processes may therefore be controlled to occur “in tandem” in both traverse and normal directions, unlike other models where specialized designs allow only diffusion within a single plane. Many example embodiments described herein employ geometrical meniscus-pinning features to facilitate direct fluid communication in both lateral and vertical directions, as an improvement over the conventional use of membranes for generating hydrogel interfaces, thereby providing a platform that is readily manufacturable and potentially cost-saving. For example, as described above, thermoplastic layers may be employed to enable stacking, alignment, and bonding of layers to create monolithic plastic biomicrofluidic systems. As described above, the out-of-plane microfluidic channels allow additional nutrient supply to the gel-embedded cells, thus improving long-term viability and rendering the platform more conducive to real-time analyses.

While the preceding example embodiments have focused on a single microchannel that comes into direct fluid communication with at least one laterally-adjacent microfluidic channel and a vertically-adjacent microfluidic channel, the present disclosure is not intended to be limited to such an implementation. Indeed, as described in detail below, the preceding example implementations may be expanded in the lateral and/or vertical direction, thereby providing more complex designs involving arrayed microchannels and perfusion microchannels that contact multiple adjacent gel structures.

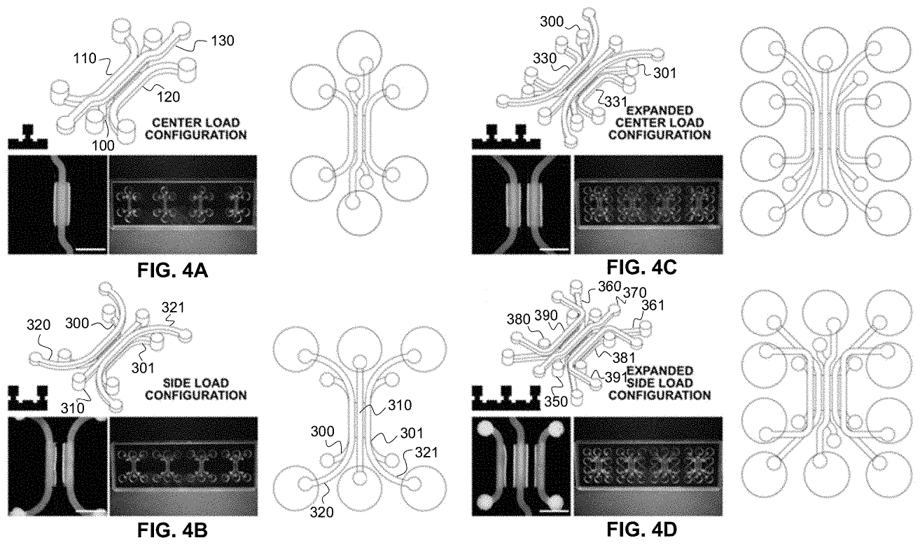

For example, the example center-load configuration described above, shown in FIG. 4A, which consists of a central microfluidic channel 100 in the lower plane that is connected to horizontally adjacent microfluidic channels 110, 120 and vertically adjacent microfluidic channel 130, may be considered a base structure or building block for creating an extended arrayed structure that is repeated laterally.

FIG. 4B shows an example side-load device configuration in which two lateral microfluidic channels 300, 301 are provided laterally on either side of, and are in direct fluid contact with, a central microfluidic channel 310, such that the microfluidic channels 300, 301 and 310 reside within a common horizontal planar region. Each of the lateral microfluidic channels 300, 301 is in direct fluid communication with a respective vertically-offset microfluidic channel 320, 321 that lies in a second horizontal planar region. Geometrical meniscus-pinning features are provided along the contact regions between each laterally and vertically adjacent pair of microfluidic channels. Such a device can be employed, for example, to culture two separate cell-containing gel structures residing in the lateral microfluidic channels 300, 301 using the central microfluidic channel 310 as a common perfusing channel, and also using vertically-offset microfluidic channels 320, 321 as perfusion channels.

Another example configuration is illustrated in FIG. 4C, which shows, relative to the configuration shown in FIG. 4B, the addition of two outer lateral microfluidic channels 330, 331 that are provided laterally on either side of, and are in direct fluid contact with, the two lateral microfluidic channels 300, 301. The additional lateral microfluidic channels can be employed as additional lateral perfusion channels when the lateral microfluidic channels 300, 301 are filled with a gel, such that the lateral microfluidic channels 300, 301 are each contacted, on either lateral side thereof, by lateral perfusion microfluidic channels.

Yet another example device configuration is illustrated in FIG. 4D. This example device configuration is an extension of the configuration shown in FIG. 4A. A central microfluidic channel 350 is brought into lateral fluid communication, on both lateral sides thereof, with a first set of lateral microfluidic channels 360, 361, such that the microfluidic channels 350, 360 and 361 reside within a common horizontal planar region. The central microfluidic channels 350 is also in direct fluid communication with a first vertically-offset microfluidic channel 370 that lies in a second horizontal planar region. A second pair of lateral microfluidic channels 380, 381 are also provided such that they reside on the outer lateral sides of the first set of microfluidic channels 360, 361, and are in direct fluid contact with the lateral microfluidic channels 360, 361, respectively. Each microfluidic channel of the second pair of lateral microfluidic channels 380, 381 is in direct fluid communication with a respective additional vertically-offset microfluidic channel 390, 391 that lie in a second horizontal planar region such that channel 390 and 391 are in the same second horizontal planar region as channel 370. Geometrical meniscus-pinning features are provided along the contact regions between each laterally and vertically adjacent pair of microfluidic channels. Such a device can be employed, for example, to culture three separate cell-containing gel structures residing in the lateral microfluidic channels 350, 380 and 381 using the first and second pairs of lateral microfluidic channels 360, 361 as shared lateral perfusing channels, and also using the three vertically-offset microfluidic channels 370, 390 and 391 as perfusion channels.

As noted above, in general, the elongate fluid communication portions at which a given microfluidic channel is brought into direct fluid communication with an (laterally or vertically) adjacent microfluidic channel need not spatially overlap with one another for all adjacent microfluidic channels of the device.

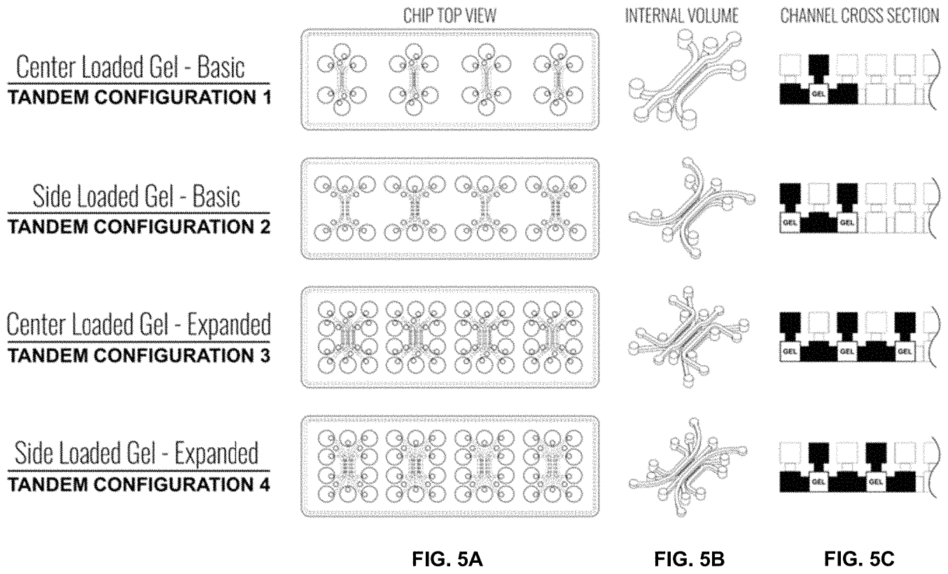

FIGS. 5A and 5B illustrates example fluidic chip designs with multiple fluidic devices per chip based on the example device configurations shown in FIGS. 4A-4D, with FIG. 5A showing an overhead view of the multi-device fluidic chip and FIG. 5B showing the internal pathways of the fluidic channels. The fluidic chips may have similar lateral dimensions to those of a conventional microscope slide, such as, for example, the fluidic chip may have a length of 75 mm plus or minus 2 mm and a width of 26 mm plus or minus 2 mm.

FIG. 5C illustrates how the various example device configurations can be spatially extended to increase the number of gel-loaded microfluidic channels and adjacent perfusion microfluidic channels. As shown in the figure, the initial pattern of microfluidic channels may be extended to form a structure in which each intermediate (i.e. not residing at either lateral end of the device) microfluidic channel residing within the first horizontal planar region is brought into direct fluid communication, via a respective lateral geometrical meniscus-pinning feature, with two laterally adjacent microfluidic channels, in where every other (i.e. every odd, or every even) microfluidic channel residing in the first horizontal planar region is also brought in to direct fluid communication, via a respective vertical geometrical meniscus-pinning feature, with a respective vertically-offset microfluidic channel residing within the second horizontal planar region that is vertically offset from the first planar horizontal region.

The example parallelization and arraying of microchannel configurations may be employed to facilitate parallel experimentation of a large library of compounds, such as for pharmaceutical discovery-type applications. For example, different compounds may be introduced into the second or third microchannels and allowed to diffuse or otherwise penetrate into the gel of the first microchannel, thus effecting biological responses on the viable cells within the gel structure. These biological responses may be measured and assayed, leading to comparisons of the efficacy of the different compounds tested in parallel, i.e., a “screening” application.

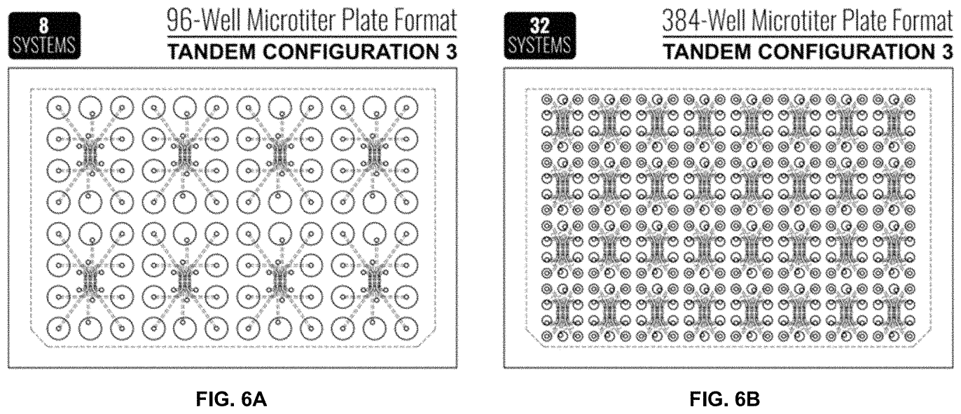

FIGS. 6A and 6B illustrate microplate implementations of an example multiplanar microfluidic device configuration, with microwells respectively associated with the perfusion microfluidic channels of the multiplanar microfluidic devices being located at array locations of a conventional microplate format (e.g. at locations corresponding to microwells in a conventional 96 well or 384 well microplate).

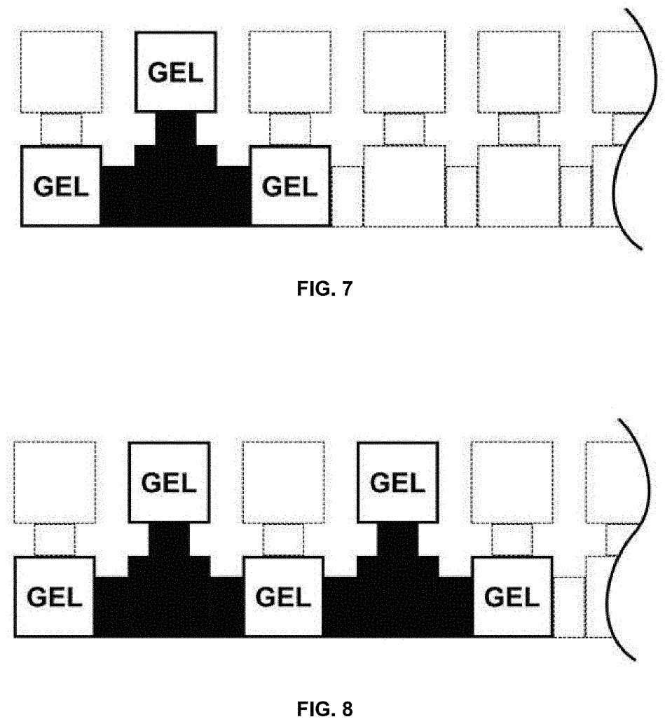

Although many of the previous example embodiments have described multiplanar microfluidic channel configurations in which some microchannels are designated or employed to form and/or house a gel (e.g. a cell-containing gel) and other microfluidic channels are designated or employed as perfusion channels, it will be understood that a given device can be utilized according to a wide variety of combinations of gel-containing microfluidic channels and perfusion microfluidic channels. For example, any of the preceding example microfluidic channel designations may be inverted such that a microfluidic channel previously designated as being employed to contain a gel may be re-designated as microfluidic channel for perfusion, and a microfluidic channel previously designated as being employed for perfusion may be re-designated as microfluidic channel for containing a gel. Example variations in microfluidic channel designation are illustrated in FIGS. 7 and 8.

The present multiplanar microfluidic embodiments may be employed to provide a generalized approach to improving diffusion within microfluidic platforms where both nutrient delivery and gas exchange may be limited and access to gel-derived supernatants is necessary. The configurable nature of channel number and spatial arrangement within the same footprint enables in vitro microenvironments to be modelled with any number of cell/hydrogel-based 3D cultures, depending on the desired outputs and goals of the end-user.

Moreover, the present multiplanar microfluidic embodiments may be configured to expose various components of the culture to open air. As described above, in some example implementations, the multiplanar systems may be split at the interface of the channel layer and port layer to expose the hydrogel through a slotted aperture for direct contact during mechanical stiffness measurements. An example of such an implementation is illustrated in FIG. 2D. In other example implementations, the vertically-adjacent perfusion microfluidic channel 120 may also be configured for vertical connection to an open air chamber 290 through a horizontal membrane 295, thus allowing for mechanical stimulation of tissue explants placed on-chip. This is illustrated in FIG. 2E. Geometrical layouts of microfluidic channels in all cases situate cell cultures adjacent to a base layer of similar thickness and optical transparency to standard cover slips, making the present multiplanar microfluidic embodiments amenable to high magnification confocal and second harmonic generation microscopy. These features have the potential to extend the capability and complexity of various biomicrofluidic and organ-on-a-chip systems.

While many of the preceding examples have employed geometric meniscus-pinning features to maintain direct fluid communication among laterally and vertically adjacent microfluidic channels while restricting transverse flow among the adjacent microfluidic channels, in other example embodiments, one or more of the geometrical meniscus-pinning features may be replaced with meniscus-pinning surface features that employ differences in hydrophobicity and/or hydrophilicity to facilitate meniscus pinning and flow restriction among adjacent microfluidic channels. For example, a surface-energy-based meniscus pinning feature may be formed by a local patterning of the substrate surface to create distinct regions of hydrophilicity and hydrophobicity. In contrast to a geometrical meniscus-pinning feature, which involves protrusions, recesses, or a combination of recesses and protrusions, a substrate surface can be patterned using masking or lithography techniques to create hydrophilic regions bordered by neighbouring hydrophobic regions. The hydrophilic regions are energetically favourable for wetting, while the hydrophobic regions are energetically unfavourable for wetting. Fluid would thus flow into and along hydrophilic regions and would establish meniscus pinning at the boundary between hydrophilic and hydrophobic regions.

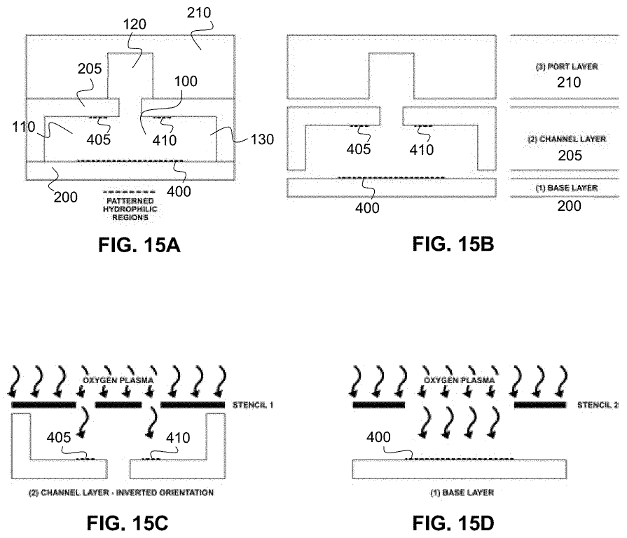

An example of a multiplanar microfluidic device in which fluid flow is restricted among adjacent microfluidic channels via surface-based meniscus pinning is illustrated in FIGS. 15A and 15B. The device includes regions of increased hydrophilicity shown at 400, 405 and 410, with the remaining surface regions having less hydrophilicity. As a consequence of this spatial patterning of hydrophilicity, a gel precursor fluid introduced into the central microfluidic channel, when it encounters the elongate fluid communication portion of the device (e.g. region 105 in FIG. 1A), will be pinned within the central microfluidic channel 100, with the difference in local hydrophilicity pinning the meniscus and preventing transverse flow into the adjacent microfluidic channels 110, 120 and 130. After the gel precursor is hardened to form a gel, the occlusion of the central portion of the device facilitates perfusion of the gel via the adjacent microfluidic channels 110, 120 and 130. FIG. 15B illustrates how the various regions of increased hydrophilicity 400, 405 and 410 can be formed within different surfaces of the first layer 200 and second layer 205 of an example multilayer device.

It will be understood that the regions of varying hydrophilicity and/or hydrophobicity can be formed according to a wide range of fabrication methods. One example technique to achieve hydrophilic-hydrophobic surface patterning is to employ a microfabricated stencil that serves as a mask on the substrate surface to be treated. The stencil can be placed in contact with the substrate surface, and the stencil-substrate assembly can be placed inside an oxygen plasma cleaner, an instrument that applies a uniform oxygen plasma treatment on the exposed substrate regions. Substrate regions covered by the solid parts of the stencil would be protected from oxygen plasma, retaining the substrate's original hydrophobicity. Substrate regions that are not covered and are positioned under the stencil openings would be exposed to oxygen plasma, rendering the surface hydrophilic. The stencil can then be removed to reveal the hydrophilic-hydrophobic pattern. A different stencil may be used for a different substrate layer, creating hydrophilic patterns on multiple substrate surfaces to achieve meniscus pinning. FIGS. 15C and 15D illustrate the formation of the regions of increase hydrophilicity 400, 405 and 410 using oxygen plasma treatment with a stencil. Furthermore, other techniques to achieve hydrophilic-hydrophobic surface patterning may be employed by using a chemical such as (but not limited to) polydopamine (PDA) to render the surface hydrophilic.

### EXAMPLES

The following examples are presented to enable those skilled in the art to understand and to practice embodiments of the present disclosure. They should not be considered as a limitation on the scope of the disclosure, but merely as being illustrative and representative thereof.

The examples below present an example fabrication method, detailed computational modeling of one particular multiplanar microfluidic configuration, cell culture experiments in a multiplanar microfluidic system, and proof-of-concept fabrication of expanded device configurations with up to 8 parallel microchannels on two different planes.

### Example 1: Example Device Fabrication Method

The device architecture utilized four separate stacked layers containing four replicate multiplanar microfluidic systems which were first developed as 3D design models using Fusion 360 (Autodesk Inc, CA, USA). The model geometries were then used to develop tool paths and set tool parameters that were collectively transmitted to machine readable G-code files, which were transferred to a 3-axis Tormach PCNC770 milling machine (Tormach, Waunakee, Wis., USA).

Acrylic sample pieces (sizes pre-determined by Fusion 360 based on the model) were cut from the cast acrylic sheets by scoring and breaking with a sharp edge. A large acrylic piece was affixed to the machining table using double sided tape and served as a “sacrificial layer” to prevent milling tools from contacting the milling stage surface during through-hole feature operations. The actual device layer sample pieces (i.e., the workpieces) were mounted on top of this sacrificial layer with double-side tape.

After setting axis references for the workpiece, the G-code was executed by the CNC machine to pattern the channel geometries on certain layers. Briefly, the four constituent layers of devices tested in this study include: (i) a blank layer at the bottom, which is cut to an identical footprint as the other layers and is otherwise unmachined to ensure high quality microscope imaging; (ii) the channel layer which is face-machined to reduce vertical separation and consists of three channels, separated by phase guides and bonded face-down to the blank layer; (iii) the port layer which houses multiple ports and a channel that is situated directly above the middle channel of the channel layer, intended for media supply; and (iv) the reservoir layer at the top which consists of reservoirs that align with port locations to increase media storage capability and thus provide sufficient nutrients for up to 24 h before media replenishment is required.

The four layers were assembled into a complete chip by pairwise thermally-assisted solvent bonding. Once layers were arranged, 100% ethanol was distributed between two of the layers by manual pipetting. These layers were then placed on the fixed platen of a Carver 3889 press (Carver Inc., Wabash, Ind., USA), with platens preheated to 70° C. The press was set to 1000 lbf (equivalent to 344 psi or 2.4 MPa for a 75 mm×25 mm microscope slide chip format) for approximately 90 s, after which the bonded layers were allowed to cool before repeating the process for other layers. Each layer also contained a retention groove (milled channel offset from the periphery of the layer) (FIG. 2B), that collected excess ethanol during bonding and improved bonding uniformity across the surface.

### Example 2: Static Cell Culture

Prior to microfluidic cell culture, devices were sterilized under aseptic conditions, first externally by spraying with 70% ethanol and then with three internal volume replacements (VRs) of 70% ethanol. Channels were then treated to another three VRs with DPBS (−/−).

After complete removal of PBS from the channels, a micropipette was used to deliver 10 μL fibronectin (FN) at a concentration of 100 μg/mL to the lower central channels only. This was used to enhance wettability and improve adhesion of the collagen gel in subsequent steps. After incubation at 37° C. for 30 min, a 7.4 pH mixture of 2.5 mg/mL collagen was added to the lower central channels and allowed to polymerize for 1 h at 37° C. For replicates containing embedded pancreatic stellate cells (PSCs), cells were harvested and resuspended in cold DPBS (5×) (1:2 dilution of DPBS (10×) in distilled water) just prior to preparation of the final collagen mixture such that the final mixture contained 1×10⁶ cells/mL.

In preparation for HUVEC monolayer formation, FN at a concentration of 100 μg/mL was then placed in the left channels of the devices and again incubated for 30 min at 37° C. During this time, HUVEC cells were re-suspended at a density of 5×10⁶ cells/mL. Immediately after removal of the FN, 10 μL of HUVEC cell suspension was added to the left channel inlet and allowed to fill the channel. The devices were then incubated at 37° C. for 2 h before EGM media was added to the channels and reservoirs.

Cells cultures were maintained in their respective media in an incubator (37° C., 95% RH, 5% CO₂) until ready for experimentation. HUVECs in EGM supplemented with 1% (v/v) Pen/Strep were harvested between passages 5 and 8. HPaSteCs in SteCM were harvested between passages 3 and 5.

### Example 3: Immunostaining of HUVEC Monolayers

After 72 h, media was removed from both left and right channels of the devices, after which the channels were subjected to three VRs of warm DPBS (+/+). Cells were then fixed with methanol at −20° C. for 10 min. The channels then underwent a further three successive VRs with cold DPBS (−/−) in preparation for staining. HUVECs in the devices were first permeabilized with 0.1% (v/v in DPBS (−/−)) Triton X-100 for 5 min, after which channels were treated to three successive VRs with DPBS (−/−). Channels were filled with blocking buffer (1% bovine serum albumin (BSA) w/v in DPBS (−/−)) and incubated at room temperature for 30 min. After removal, primary antibody solution (rabbit anti-VE-cadherin, 3 μg/mL in 1% BSA) was added to the HUVEC lumen channels and incubated for 4 hr at room temperature. At this time, single-well plates containing devices were sealed with parafilm to reduce evaporation. The secondary antibody solution (goat anti-rabbit-Alexa Fluor® 568, 1:100 in 1% BSA) was then added to the channel (protected from light) after three successive VRs with DPBS (−/−). Devices were incubated at room temperature for 30 min, triple washed once again, and monolayers were counterstained with Hoechst 33342 nucleic acid stain for 5 min at room temperature. Channels were finally triple washed once more with DPBS (−/−) before wrapping single-well plates in foil and storing at 4° C. prior to imaging within 24-48 h.

### Example 4: Dextran Diffusion Rate Measurement

Basic two-channel devices (in SP) were prepared with the right channel containing polymerized type I collagen and the left channel with or without the presence of endothelial monolayers. Devices were imaged after 72 h using a live cell imaging system (EVOS®—FL Auto, see Imaging below). A 3-h time lapse (1 min frequency) was set to start immediately after media in the left channels of both the control and monolayer conditions were replaced with 20 μL of media containing 50 μM 10-kDa FITC-Dextran. Image stacks were processed in ImageJ. Normalized average fluorescence intensity at a centralized region-of-interest (ROI) in each channel was plotted against time over the 3-h interval.

### Example 5: Stellate Cell Viability Assay

To compare single-plane (SP) designs with dual-plane (DP) designs, PSCs were cultured embedded in type I collagen as previously described. The SP design was a three-channel system with no top channel, while the DP design had the same three-channel system on the lower plane with an additional top channel, situated directly above the middle channel of the lower plane. Control conditions were loaded with the collagen mixture only and both were allowed to polymerize for 1 hr before addition of a 50/50 mixture of EGM and SteCM media in the side channels. SteCM was also added to the top channel of DP systems. Viability assays were performed on replicate batches both at the 24 h and 72 h mark by adding 20 μL aliquots from 1 mL of serum-reduced media containing 1 μL of Calcein AM and 2 μL of Ethidium Homodimer. Devices were incubated at 37° C. for 1.5 h to ensure sufficient diffusion into the hydrogel. Cell-embedded hydrogel channels were then imaged immediately after incubation and ImageJ was used to produce maximum intensity composite projections from 10-slice z-stacks of each ROI within the hydrogel channels.

### Example 6: Design Rationale and Testing

To illustrate the transport and functionality of multiplanar microfluidic systems, the present study focused on the most basic center-load configuration consisting of three in-plane microchannels (left channel—LC, middle channel—MC, right channel—RC) in the lower channel layer and an additional fourth microchannel in the upper channel layer, situated directly above and connected to the MC of the lower channel layer. Separating all adjacent channels were elongate meniscus-pinning edge feature, which were chosen because they were reliable at achieving stable fluid pinning, easier to micromachine than microposts, and offered a greater area for diffusive flux compared to microposts. In this configuration, a reservoir top layer was used for static culture conditions, but the design allows for this layer to be replaced with a secondary port layer to facilitate world-to-chip connections when shear flow and media perfusion are desirable. This multiplanar design in turn allowed vertical stacking of microfeatures, creation of multiple orthogonal meniscus-pinning feature pairs, and ultimately more complex channel geometries (FIG. 2B).

PMMA was selected as a device material to avoid PDMS-associated issues and open up the present multiplanar microfluidic devices to the possibility of manufacturing at scale. Additionally, it was found that PMMA was attractive due to its ease of machining, cleaning and bonding, its optical clarity, and its relative bioinertness compared to other commonly used materials. Of the variety of thermoplastic fabrication techniques available to us, micromilling was chosen as it allows rapid iteration and conversion from computer-aided design (CAD) models to finished device, and can be scaled to medium volume production when integrated with other processes such as hot embossing. Once the layers were machined, the solvent bonds between each layer of the design were resistant to spontaneous or humidity-induced delamination, but still enabled the separation of layers when desired. This reversibility permitted perfusion and shear flow at relatively high flow rates (and by extension high pressures), but also maintained the ability to separate layers and access cell and tissue samples for analysis when needed.

### Example 7: Fabrication and Characterization of Elongate Meniscus-Pinning Edge Features

The dimensions of elongate meniscus-pinning edge feature for the present example multiplanar microfluidic designs were optimized by maximizing fluid pinning capability while factoring the constraints of micromilling. Heights and widths of the meniscus-pinning edge features were minimized to maximize the cross-sectional area for diffusive flux, but heights that are too small in comparison to overall channel heights will fail to establish sufficient interfacial tension at the surface, thus leading to fluid overflow into adjacent channels. Additionally, thinner the meniscus-pinning edge features also bring adjacent channels closer together, but they reduce structural stiffness and thus become increasingly prone to fracture due the cyclic loading of the micromilling tool during the milling process.

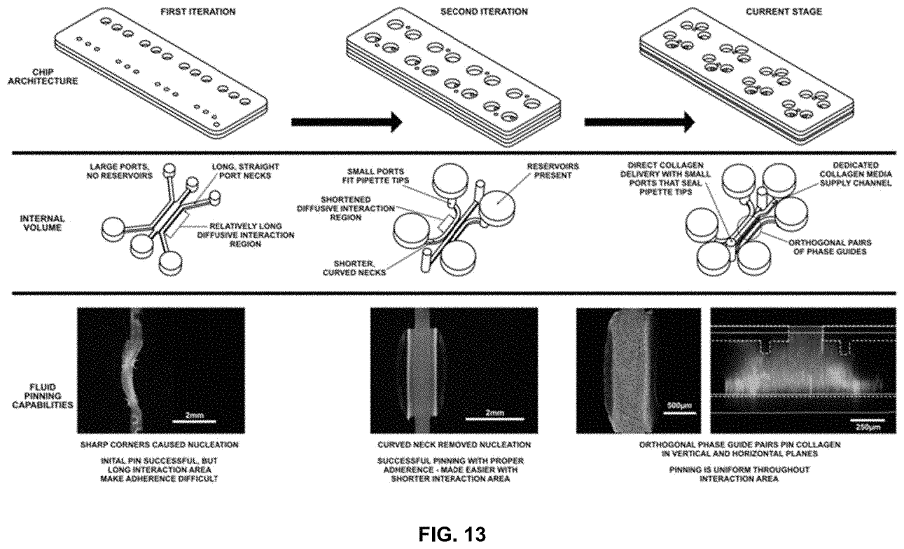

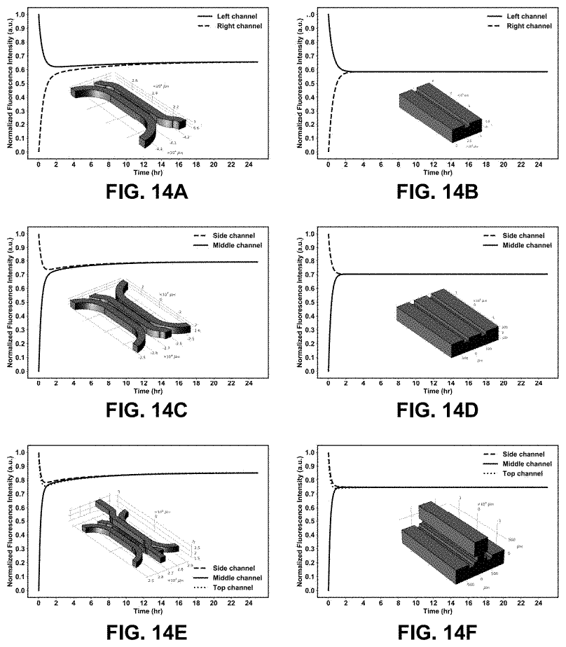

To satisfy these conditions, channel and the meniscus-pinning edge feature geometries in multiplanar microfluidic devices were empirically optimized over multiple iterations to improve hydrogel pinning quality and repeatability. It was found that bubble nucleation occurring within the hydrogel was significantly reduced by removal of sharp corners in the channels (FIG. 13). An overall reduction in the length of the column of hydrogel being pinned by the meniscus-pinning edge features also significantly improved pinning repeatability, with a final length of 3 mm ensuring >95%.

Different height-to-width ratios for pairs meniscus-pinning edge features were tested for the current design and results indicated high accuracy and precision in machinability (FIG. 3C). When tested with 2.5 mg/mL type I collagen mixture, it was found that pinning was more consistent across the entire range of heights for the 200-μm wide the meniscus-pinning edge features in contrast to the 100-μm wide meniscus-pinning edge features, where failures were observed for heights below 100 μm (FIG. 3D). Based on the selected channel height of 500 μm and the current milling technology employed, it was deduced that for 100-μm wide meniscus-pinning edge features, heights below 75 μm had unreliable surface finishes due to fracturing and thus pinning was not repeatable. As a result, 200-μm wide meniscus-pinning edge features were selected. A height of 125 μm proved to be most repeatable after numerous loading tests in multiplanar microfluidic devices and also coincides with a 25% fraction of overall channel height.

### Example 8: Simulations Via Computational Transport Model

To understand multidirectional diffusion in the present example multiplanar microfluidic designs, a computational transport model was developed in COMSOL that uniquely accounted for (i) porous media transport through the hydrogel and (ii) barrier transport through endothelial monolayers.

A simplified two-channel model of the diffusion testing device was first set up in COMSOL by importing the same 3D modelling data used for CNC fabrication. The hydrogel channel was designated as a porous media flow domain while the adjacent channel was designated as a free-flow domain. Dilute species mass transport physics was added to the entire system with the appropriate boundary conditions at the inlets and outlets as well as an initial concentration of 50 μM of dilute species in the channel adjacent to the hydrogel.

A parametric sweep was then performed for porosity ranging from 50% to 100% and diffusivity ranging from 1.6-2.5×10⁻¹⁰ m²/s. Optimal parameter values were determined by minimization of the sum of squared residuals (SSR) between each simulation condition curve and the experimental plots.

Once the model-fitted diffusivity and porosity values were determined, the values were used in a secondary simulation stage involving a diffusion barrier as an added internal boundary condition at the interface between the two channels. This condition was intended to simulate the presence of cell monolayers specifically with respect to impeding diffusion of the dilute species.

Similarly, the barrier diffusivity was also simulated by parametric sweeping over a range (0.5-2.5×10⁻¹² m²/S) and an optimal value was selected by SSR minimization when compared to the experimental results involving the presence of monolayers in the left channels.

Finally, DP and SP designs (defined above in Stellate cell viability assay) were modelled in COMSOL under similar conditions using the optimized parameters determined by experimental validation. Simulations were performed to examine nutrient diffusion toward collagen-embedded cells (i.e., inward diffusion from peripheral channels toward the collagen channel), and also to study cell-secreted soluble factors and their diffusion from the gel to the side channels (i.e., outward diffusion from collagen channel toward peripheral channels).

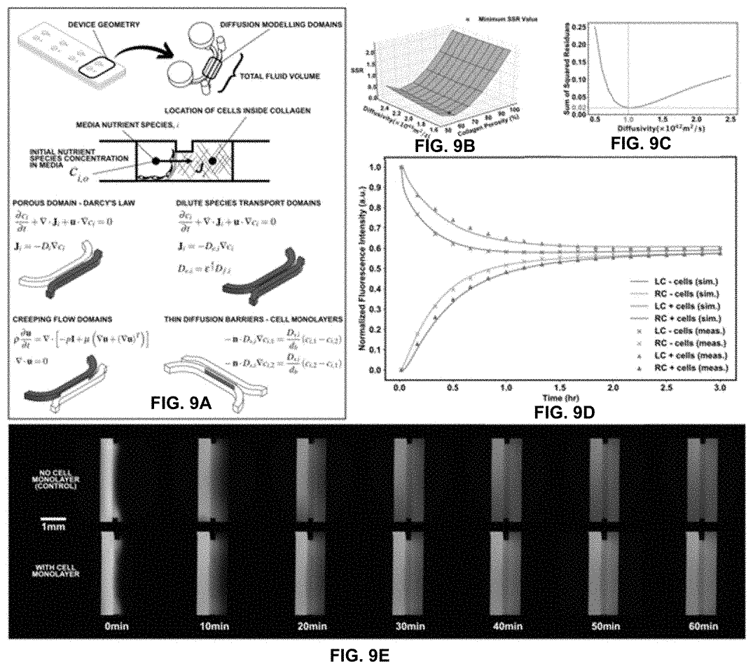

As noted above, simulations were first performed for a more basic non-multiplanar two-channel design that could then be easily verified by experiments. The two-channel system was simulated to assess transport of 10-kDa FITC-Dextran from the non-porous “free flow” left channel to the “porous medium flow” right channel, which modelled a collagen-based hydrogel (FIG. 9A). For simulations involving endothelial monolayers on the gel interface, the monolayer was modelled as a “thin diffusion barrier”. An important consideration is that diffusivity through a barrier is lower than diffusivity in a “free flow” aqueous solution for the same molecular species. In the present simulations, collagen porosity E, FITC-Dextran diffusivity in aqueous solution D_{d,aq}, and effective FITC-Dextran diffusivity through the cell monolayer, D_{d,e} were treated as independent parametric values that can be fitted to the experimental data a posteriori.

In the present experiments, normalized fluorescence intensity values were extracted from a 3-h timelapse of FITC-Dextran transport in a fabricated two-channel system (FIG. 9D, 9E). First, to determine physical values for D_{d,aq} and E (FIG. 9B), parametric sweeping was performed across a range of numerical values for D_{d,aq} and E in COMSOL and fit the diffusion transport curves for each parameter set to the normalized experimental data for the no-cell (control) condition. This first simulation step resulted in D_{d,aq}=170 μms⁻¹ and ε=0.60, which was consistent with literature. A porosity of 60% for the hydrogel is relatively low and can be attributed to possible dehydration and micro-bubble formation over the 3-h period, leading to reduced effective volume of the porous medium. In addition, concentration curves plateaued by the 3-h timepoint, but a persistent gap between the concentration curves indicated that equilibrium was not completely reached at 3 h. Interestingly, it was found that the surrounding channel and reservoir volumes played a significant role in the transport model, and that neglecting these features in the model geometry led to artifacts that do not match the experimental results (FIGS. 14A-14F).

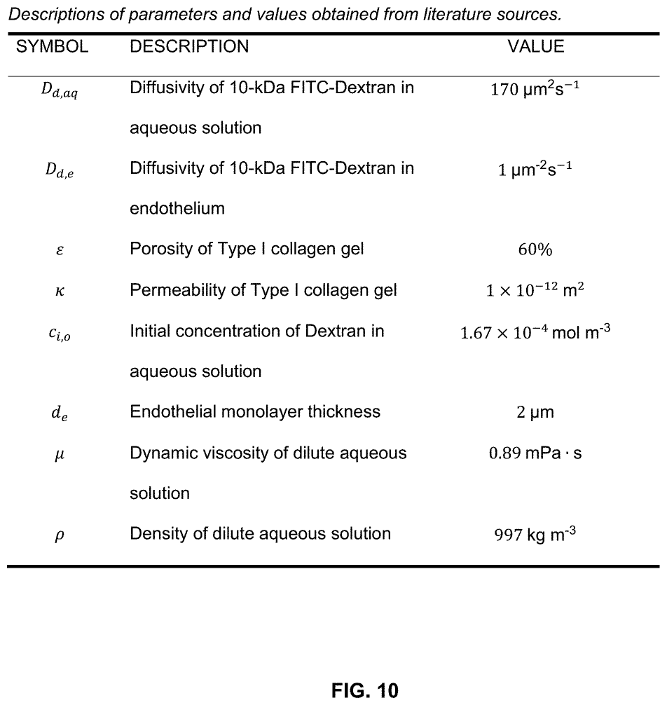

Next, to determine a value for D_{d,e}, a “thin diffusion barrier” condition was applied at the interface between the two channels and performed similar parametric sweeping using the previously fitted values for D_{d,aq} and ε. Simulated diffusion transport curves were fitted to the experimental data for the monolayer condition, resulting in D_{d,e}=1 μm s⁻¹ (FIG. 9C). While typical studies report permeability as a useful metric to quantify barrier resistance, this method of modelling assesses resistance based on an effective diffusivity (D_{d,e}). The fitted values obtained here were then used in subsequent diffusion simulations for examining the present example multiplanar microfluidic designs (FIG. 10).

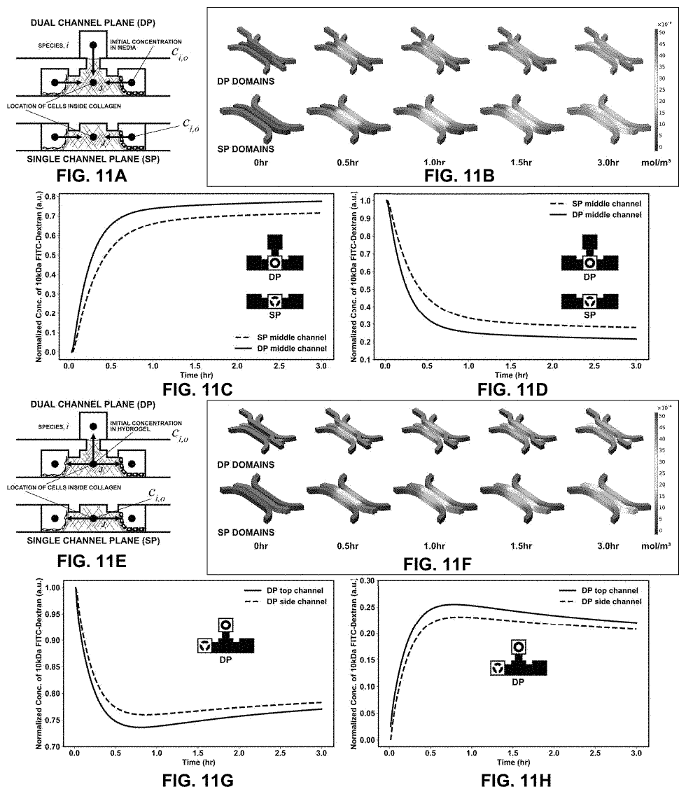

After validating the computational model and obtaining fitted values for D_{d,e}, D_{d,aq}, and ε, the model was applied to study diffusion transport in both (i) a single plane (SP) three-channel design without a top channel and (ii) a dual-plane (DP) four-channel design with a top channel (i.e., multiplanar microfluidic). In the first scenario, cell monolayers were modelled at the LC/MC and RC/MC interfaces with maximum initial species concentration c_{i,0} in the peripheral channels (FIG. 11A). The purpose of this scenario was to examine the rate of diffusive flux into the hydrogel, which serves as a model for the transport of nutrients from fresh media in peripheral channels toward the cells embedded in the hydrogel. This allows us to understand and predict the effect of the 3D interconnected channel arrangement on gel-embedded cell viability, where a top layer provides a media-filled channel above, as opposed to gel-embedded cell viability based solely on diffusion via laterally adjacent channels alone. It was found that the spatial distribution of species concentration over time changed rapidly in the lateral direction across the gel interfaces, as compared to the longitudinal direction from reservoir to channel center where the concentration changed more gradually. (FIG. 11B). When the rates of concentration increase in the porous medium of the MC were examined, it was found that the DP design had faster diffusion rates compared to the SP design (FIG. 11C), suggesting that more nutrients would be delivered to gel-embedded cells in the DP design. In addition, when rates of concentration decrease (i.e., nutrient depletion) were compared between the top channel and the side channels of the DP design, it was found faster depletion in the top channel compared to the depletion in the side channels, which was expected due to the presence of cell monolayers that acted as barriers to diffusion into the side channels (FIG. 11D).

In the second scenario, cell monolayers were again modelled at the LC/MC and RC/MC interfaces, but maximum c_{i,0} was prescribed within the MC porous medium instead of the peripheral channels (FIG. 11E). The purpose of this scenario was to examine the rate of diffusive flux out of the hydrogel, which serves as a model for the transport of secreted factors by gel-embedded cells into the neighbouring channels. This allowed the assessment of the feasibility of multiplanar microfluidic devices for various endpoint analyses involving secreted factors, where analytes can be extracted from each channel and used to decouple the secretory profiles of the gel-embedded culture from those of the monolayer cultures. Spatial distribution of species concentration over time was consistent with previous findings of the present inventors for the first scenario (FIG. 11F). In addition, outward diffusive flux was higher in the DP design than the SP design (FIG. 11G), while species concentration increased faster in the top channel than in the side channels of the DP design (FIG. 11H).

Taken together, these simulations showed that the top channel has unique benefits that include: (i) faster rate of supplying gel-embedded cells with more nutrients, (ii) providing access to secreted factors diffusing from the hydrogel in a manner that is unimpeded by cell monolayers, and (iii) enabling the collection of secreted factors as cell culture supernatants in the top channel for the purpose of downstream profiling and analyses. All of these benefits are derived from enhanced diffusion, enabled by having a second plane of microchannels in the multiplanar microfluidic architecture. Note that the enhanced diffusion shown here is specific to the cross-sectional areas chosen in the simulations, and it is thus expected even greater enhanced diffusion in cases where the lateral cross-sectional areas for flux are further reduced and the normal cross-sectional areas for flux (into the top channel) are further increased.

### Example 9: Collagen-Embedded Stellate Cell Viability in DP and SP Designs

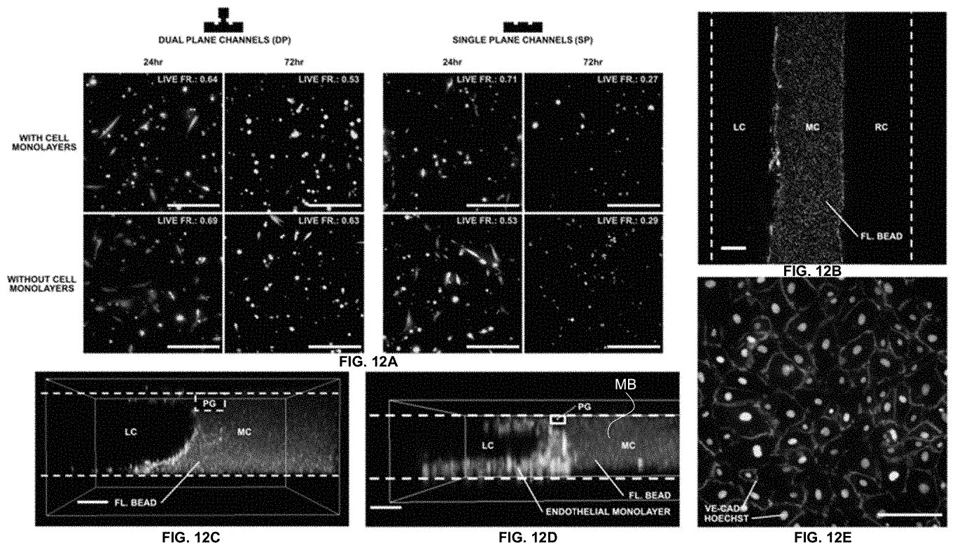

To test whether enhanced diffusion of the DP design causes a real biological effect on cultured cells, PSCs were embedded in a type I collagen gel mixture within the MC of both DP and SP designs and compared their viability over several days, both in monoculture and in coculture with HUVEC monolayers on gel interfaces (FIGS. 12A-12E). In both monoculture and coculture conditions, cell viability decreased significantly more in the SP design (44% for monoculture, 24% for coculture) than in the DP design (11% for monoculture, 6% for coculture) from 24 h to 72 h (FIG. 12A). This was expected given the simulated predictions of enhanced diffusion in multiplanar microfluidic designs. Furthermore, cocultures in both SP and DP designs showed greater decrease in cell viability over time, which was consistent with the simulated predictions that cell barriers lower the rates of diffusion. Together, the results support the hypothesis that open TCs in multiplanar microfluidic designs lead to significantly greater nutrient supply to the MC, especially in cases where cell barriers that are present on gel interfaces further restrict nutrient delivery in the lateral plane. In these experiments, baseline cell viability at 24 h was only ˜50-70% for all conditions, likely attributable to poor gas permeability in thermoplastic devices. Unlike PDMS, thermoplastics restrict the gas exchange to media in the channels and reservoirs rather than allowing diffusion through the bulk material. As a result, oxygen deprivation in the first 24 h may lead to lower cell viability in general. To visualize cell barriers at gel interfaces, fluorescent beads were loaded to confirm successful pinning of the collagen gel in the MC via the elongate meniscus-pinning edge features (FIG. 12B), and stained HUVEC monolayers for VE-cadherin, a key intercellular junction protein (FIG. 12C). Surface tension effects of unpolymerized collagen resulted in a concave gel interface on which confluent HUVEC monolayers were observed with VE-cadherin localization around endothelial cell borders (FIG. 12D-12E).

### Example 10: Mathematical Modeling of Diffusion

### Quantification of 10-Kda FITC-Dextran Diffusion in Two-Channel Geometry:

Time lapse images for the 3-h period were converted to stacks in ImageJ and used to generate 8-bit values for average pixel intensity of ROIs located at the center of the left and right channels respectively. The original dataset (x), was then converted to a normalized dataset (z), by performing the following function on all elements within x:

$${z}_{i} = \frac{{x}_{i} - min ( x )}{max ( x ) - min ( x )}$$

The same operation was also performed on all simulated datasets produced by COMSOL. Left channel and right channel data for both measured and simulated species transport were then plotted from the normalized datasets and used in all further analyses.

### Model of Creeping Flow in Free-Flow Domains:

In general, fluid flow within the control volume is governed by the Navier-Stokes equations for incompressible fluid motion:

$$ρ \frac{\partial u}{\partial t} + ρ ( u \cdot ∇ ) u = ∇ \cdot [ - p I + T ] + F \qquad ( 1 )$$

$$∇ \cdot u = 0 \qquad ( 2 )$$

$$T = μ ( ∇ u + {( ∇ u )}^{T} ] \qquad ( 3 )$$

where ρ is the fluid density, μ represents the dynamic viscosity, u is the velocity field vector, p is pressure, T is the viscous stress tensor and F is the per-unit-volume body force vector. Given the characteristic velocity and length scale of the system, low Reynolds number laminar flow is valid (Re<<1), and the convective term above may be neglected. Additionally, in the present case, body forces were considered negligible. This results in simplified Stokes flow within the channels:

$$ρ \frac{\partial u}{\partial t} = ∇ \cdot [ - pI + μ ( ∇ u + {( ∇ u )}^{T} ) ] \qquad ( 4 )$$

$$∇ \cdot u = 0 \qquad ( 5 )$$

### Model of Porous Media Flow:

For transport within the hydrogel compartment, Darcy's Law of permeability was used to treat the hydrogel as a homogeneous porous medium (defined as a “matrix domain” in COMSOL):

$${u}_{D} = - \frac{κ}{μ} ∇ p \qquad ( 6 )$$

$$\frac{\partial }{\partial t} ( ρ ε ) + ∇ \cdot ( ρ {u}_{D} ) = {Q}_{m} \qquad ( 7 )$$

where u_{D} is the Darcy velocity field vector within the porous domain (often referred to as the superficial velocity), κ is the Darcy permeability of the porous medium, ε is the porosity, and Qₘ is the mass source term. Equation (7) above is essentially the continuity equation as it pertains to porous media.

### Model of Mass Transport of Diluted Species in Free-Flow Domains:

For a given nutrient species, i, a mass balance can be performed:

$$\frac{\partial {c}_{i}}{\partial t} + ∇ \cdot {J}_{i} + u \cdot ∇ {c}_{i} = {R}_{i} \qquad ( 8 )$$

$${J}_{i} = - {D}_{i} ∇ {c}_{i} \qquad ( 9 )$$

where Jᵢ is the diffusive mass flux of species i, u is the mass-averaged velocity vector, cᵢ is the concentration of the species, Dᵢ is the species diffusivity, and Rᵢ is the reaction rate of the species. Note in this case that no reaction takes place, which results in the following system to define mass transport in the free-flow domains:

$$\frac{\partial {c}_{i}}{\partial t} + ∇ \cdot {J}_{i} + u \cdot ∇ {c}_{i} = 0 \qquad ( 10 )$$

$${J}_{i} = - {D}_{i} ∇ {c}_{i}$$

### Model of Mass Transport of Diluted Species in Porous Domains:

While the previous set of equations is true for a free-flow (non-porous medium) domain, the equations require modification for a porous medium domain because diffusivity of a molecule in porous media must be lower than its diffusivity in free-flow. This creates a distinction between effective diffusivity and free-flow diffusivity of a species and real concentration gradient as compared to apparent concentration gradient. Millington and Quirk¹ have shown considerable accuracy in accounting for this by scaling the diffusivity using a ratio of the total porosity and tortuosity factor, τ of the porous medium:

$$\frac{\partial {c}_{i}}{\partial t} + ∇ \cdot {J}_{i} + u \cdot ∇ {c}_{i} = {R}_{i} + {S}_{i} \qquad ( 11 )$$

$${J}_{i} = - {D}_{e , i} ∇ {c}_{i} \qquad ( 12 )$$

$${D}_{e , i} = \frac{ε}{τ} {D}_{f , i} \qquad ( 13 )$$

For porous solids with steady diffusive flow, Millington² also demonstrated that this tortuosity factor and total porosity can be related as shown:

$$τ = {ε}^{- \frac{1}{3}} \qquad ( 14 )$$

resulting in the following:

$${D}_{e , i} = {ε}^{\frac{4}{3}} {D}_{f , i} \qquad ( 15 )$$

In the above equations, Sᵢ is an additional source/sink term for species i, D_{e,i} is the effective diffusivity in porous media, and D_{f,i} is the actual diffusivity of molecular species in an open fluid. Similarly, as above, Equation (11) reduces to Equation (10). The resulting set then describes mass transport of species i, in the porous media channel:

$$\frac{\partial {c}_{i}}{\partial t} + ∇ \cdot {J}_{i} + u \cdot ∇ {c}_{i} = 0$$

$${J}_{i} = - {D}_{e , i} ∇ {c}_{i}$$

$${D}_{e , i} = {ε}^{\frac{4}{3}} {D}_{f , i}$$

### Tissue Barrier Function of Mass Transport:

Cell monolayers separating the side channels from the porous media in the middle channel can significantly affect lateral species transport. This highlights a key difference of these channels compared to the additional top channel (TC) of the DP design. The effects of diffusion due to the permeability of the monolayers can be modelled using a thin diffusion barrier separating the free-flow domain (subscript 1) and the porous domain (subscript 2):

$$- n \cdot {D}_{s , i} ∇ {c}_{i , 1} = \frac{{D}_{s , i}}{{d}_{b}} ( {c}_{i , 1} - {c}_{i , 2} ) \qquad ( 16 )$$

$$- n \cdot {D}_{s , i} ∇ {c}_{i , 2} = \frac{{D}_{s , i}}{{d}_{b}} ( {c}_{i , 2} - {c}_{i , 1} ) \qquad ( 17 )$$

In the equations above, d_{b} is the barrier thickness. Equations (16) and (17), along with the other equations highlighted in this section, provide a general governing set of equations for mass transport in the multiplanar microfluidic device.

### Example 11: Reflected Light Confocal Imaging of Collagen Fibers In Situ

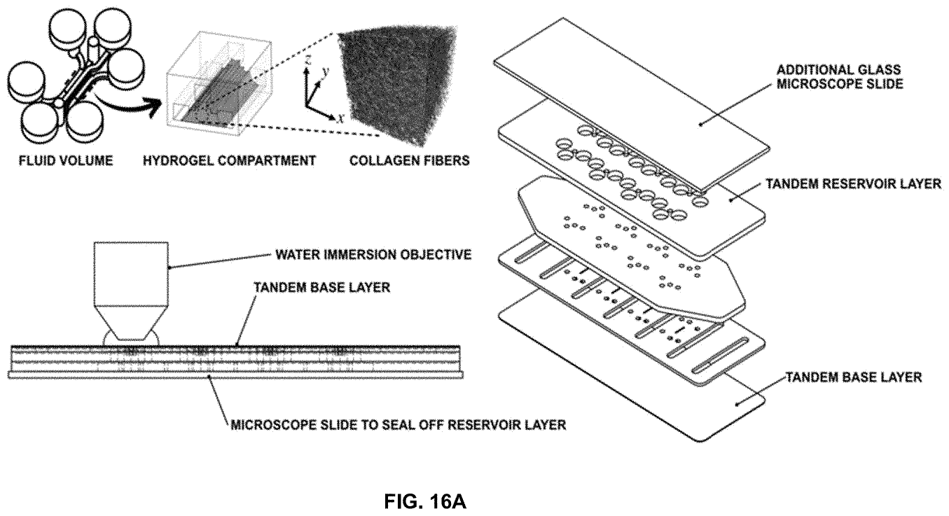

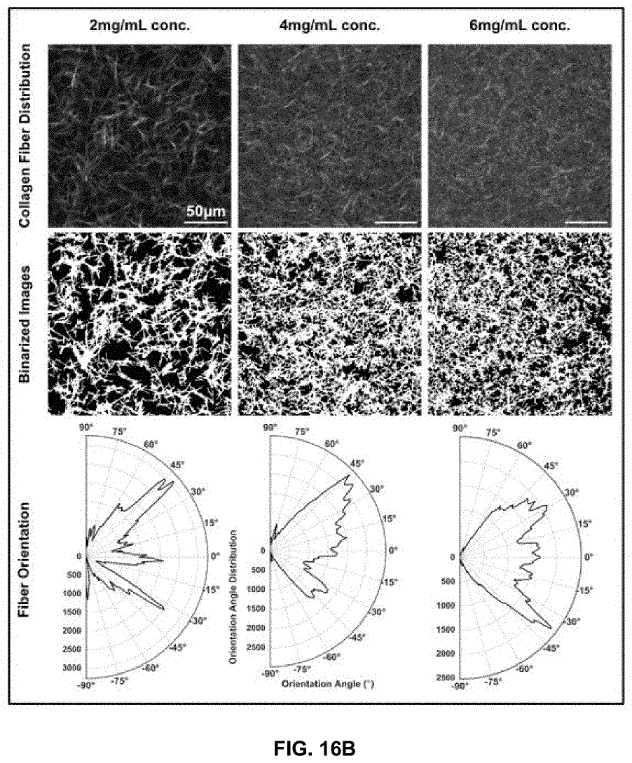

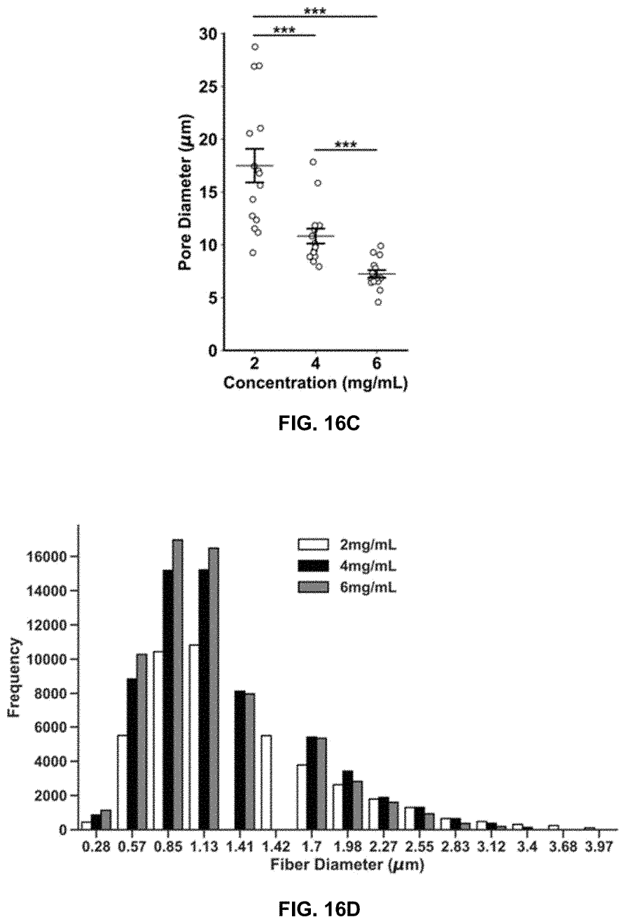

Devices containing polymerized Type I collagen in the lower middle channel were sealed with a 75 mm×25 mm microscope slide (VWR International, Radnor, Pa., USA), inverted and placed on the stage of an upright Zeiss LSM710 confocal microscope (White Plains, N.Y., USA), as shown in FIG. 16A. Reflected light was used to acquire z-stacks of collagen fiber images through the 200 μm-thick base layer with a water immersion objective at 20× magnification (Plan-Apochromat, 20×/1.0 DIC (UV) VIS-IR). Stacks were 300-400 μm thick with 5 μm step-size. Images were then processed in Imaris (Oxford Instruments, Abingdon, UK) to characterize samples in terms of pore size, fiber density, length and alignment, as shown in FIGS. 16B, 16C and 16D.

### Example 12: On-Chip Stiffness Measurements Using Atomic Force Microscopy (AFM)

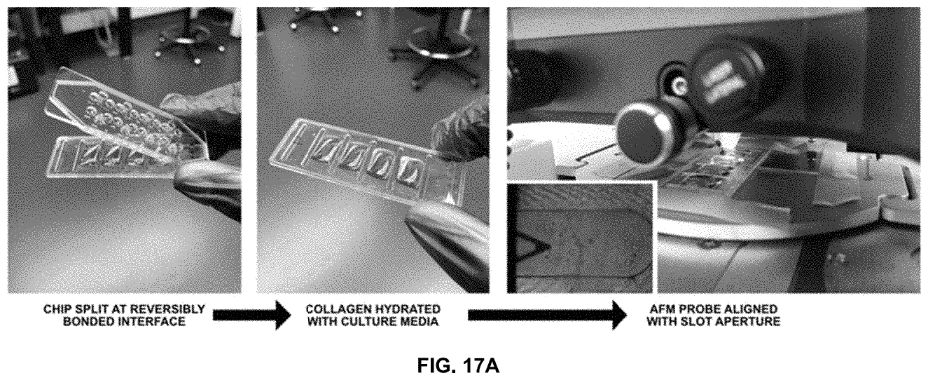

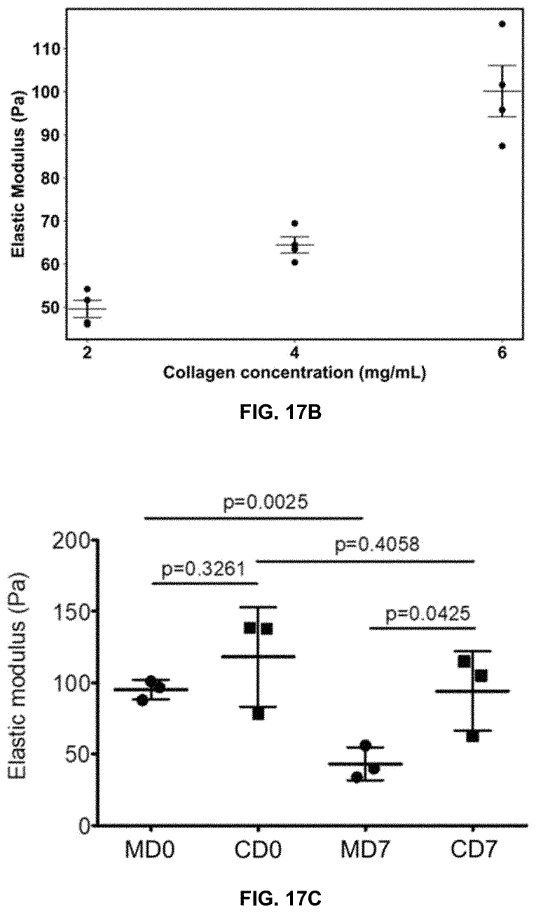

A reversibly bonded interface between upper- and lower-layer pairs was created using thermally assisted solvent bonding (60% ethanol for 1.5 min under 1000 lbf). Each of the two lower and upper layers was permanently bonded using previously published solvent bonding protocols. This allowed the base/channel layer pair to be separated from the port/reservoir layer pair to expose the gel compartment for direct access by the AFM probe, as shown in FIG. 17A.

Elastic moduli of the collagen gels were measured in a hydrated state by nanoindentation using a JPK atomic force microscope (Bruker JPK NanoWizard 4, Cambridge, UK) in force spectroscopy contact mode (FIG. 17A). Tip-less silicon nitride cantilevers (Bruker, MLCT-O10, with a nominal spring constant of 0.05 N/m) was functionalized using 10 μm radius spherical polystyrene beads (Phosphorex Inc. Hopkinton, Mass., USA) and calibrated using the contact-based thermal tune method. Elastic moduli were estimated from the force-deflection extend curves for 60 independent measurements per sample, analyzed based on the Hertz/Sneddon model using JPK Data Processing software (version 6.3.11).

Devices were rotated by 90 degrees to align the exposed slot apertures to the direction of the cantilever and thus remove any edge-based interference (FIG. 17A, inset). Elastic moduli were obtained for three concentrations of type I collagen gel: 2 mg/mL, 4 mg/mL and 6 mg/mL, as shown in FIG. 17B. This method was also used to determine the change in mechanical stiffness of gels embedded with PSCs at initial seeding (MD0) and at day 7 (MD7) and cocultured with pancreatic tumor cells at the same time points (CD0, CD7), as shown in FIG. 17C.

The specific embodiments described above have been shown by way of example, and it should be understood that these embodiments may be susceptible to various modifications and alternative forms. It should be further understood that the claims are not intended to be limited to the particular forms disclosed, but rather to cover all modifications, equivalents, and alternatives falling within the spirit and scope of this disclosure.

Therefore what is claimed is:

1. A microfluidic device comprising:
a first microfluidic channel extending within a first horizontal planar region;
a second microfluidic channel extending within said first horizontal planar region, said second microfluidic channel residing laterally adjacent to said first microfluidic channel along a first portion of said first microfluidic channel such that said second microfluidic channel is in direct lateral fluid communication with said first microfluidic channel along said first portion of said first microfluidic channel in the absence of a membrane therebetween;
a third microfluidic channel extending within a second horizontal planar region, said second horizontal planar region being vertically offset from said first horizontal planar region, said third microfluidic channel residing vertically adjacent to said first microfluidic channel along a second portion of said first microfluidic channel such that said third microfluidic channel is in direct vertical fluid communication with said first microfluidic channel along said second portion of said first microfluidic channel in the absence of a membrane therebetween;
a lateral geometrical meniscus-pinning feature residing between said second microfluidic channel and said first microfluidic channel within said first portion of said first microfluidic channel, said lateral geometrical meniscus-pinning feature being configured to resist fluid flow between said first microfluidic channel and said second microfluidic channel within said first portion of said first microfluidic channel; and
a vertical geometrical meniscus-pinning feature residing between said third microfluidic channel and said first microfluidic channel within said second portion of said first microfluidic channel, said vertical geometrical meniscus-pinning feature being configured to resist fluid flow between said first microfluidic channel and said third microfluidic channel within said second portion of said first microfluidic channel.

2. The microfluidic device according to claim 1 wherein said first portion of said first microfluidic channel overlaps at least in part with said second portion of said first microfluidic channel.

3. The microfluidic device according to claim 1 wherein at least one of said lateral geometrical meniscus-pinning feature and said vertical geometrical meniscus-pinning feature comprises an elongate meniscus-pinning edge feature extending longitudinally between said first portion of said first microfluidic channel and said second microfluidic channel.

4. The microfluidic device according to claim 3 wherein said elongate meniscus-pinning edge feature comprises a meniscus-pinning ridge.

5. The microfluidic device according to claim 1 wherein said lateral geometrical meniscus-pinning feature comprises a lateral meniscus-pinning ridge and said vertical geometrical meniscus-pinning feature comprises a vertical meniscus-pinning ridge, said lateral meniscus-pinning ridge and said vertical meniscus-pinning ridge being configured to resist fluid flow in orthogonal directions.

6. The microfluidic device according to claim 1 wherein at least one of said lateral geometrical meniscus-pinning feature and said vertical geometrical meniscus-pinning feature comprises a plurality of microposts.

7. The microfluidic device according to claim 1 further comprising a first inlet port and a first outlet port in flow communication with said first microfluidic channel.

8. The microfluidic device according to claim 7 wherein said first inlet port is configured to be sealed by insertion of a pipette tip, thereby facilitating dispensing of a fluid into said first microfluidic channel from the pipette tip after insertion of the pipette tip into said first inlet port.

9. The microfluidic device according to claim 7 further comprising:
a second inlet port and a second outlet port in flow communication with said second microfluidic channel; and
a third inlet port and a third outlet port in flow communication with said third microfluidic channel;
wherein said second inlet port, said second outlet port, said third inlet port and said third outlet port reside within respective microwells.

10. The microfluidic device according to claim 1 further comprising:
a first layer;
a second layer bonded to said first layer;
wherein at least one of an upper surface of said first layer and a lower surface of said second layer comprise recessed features that define, after said first layer is bonded to said second layer, said first microfluidic channel, said second microfluidic channel, and said lateral geometrical meniscus-pinning feature;
said second layer further comprising at least one aperture defined such that said aperture resides above said first microfluidic channel after said first layer is bonded to said second layer; and
a third layer secured to said second layer, wherein a lower surface of said third layer comprises an additional recessed feature that defines, after said third layer is bonded to said second layer, said third microfluidic channel, and wherein said aperture forms, at least in part, said vertical geometrical meniscus-pinning feature.

11. The microfluidic device according to claim 10 wherein said third layer is detachably removable from said second layer to provide direct access to contents of said first microfluidic channel.

12. The microfluidic device according to claim 1 further comprising a reservoir defined above at least a portion of said third microfluidic channel, said reservoir being in fluid communication with said third microfluidic channel via a horizontal membrane residing between said reservoir and said third microfluidic channel.

13. The microfluidic device according to claim 1 wherein said lateral geometrical meniscus-pinning feature is a first lateral geometrical meniscus-pinning feature, said microfluidic device further comprising:
a fourth microfluidic channel extending within said first horizontal planar region, said fourth microfluidic channel residing laterally adjacent to said first microfluidic channel along a third portion of said first microfluidic channel such that said fourth microfluidic channel is in direct lateral fluid communication with said first microfluidic channel along said third portion of said first microfluidic channel in the absence of a membrane therebetween, said first microfluidic channel residing between said second microfluidic channel and said fourth microfluidic channel; and
a second lateral geometrical meniscus-pinning feature residing between said first microfluidic channel and said fourth microfluidic channel within said third portion of said first microfluidic channel, said second lateral geometrical meniscus-pinning feature being configured to resist fluid flow between said first microfluidic channel and said fourth microfluidic channel within said third portion of said first microfluidic channel.

14. The microfluidic device according to claim 13 wherein said vertical geometrical meniscus-pinning feature is a first vertical geometrical meniscus-pinning feature, said microfluidic device further comprising:
a fifth microfluidic channel extending within said first horizontal planar region, said fifth microfluidic channel residing laterally adjacent to said fourth microfluidic channel along a first portion of said fifth microfluidic channel, such that said fourth microfluidic channel is in direct lateral fluid communication with said fifth microfluidic channel along said first portion of said fifth microfluidic channel in the absence of a membrane therebetween, said fourth microfluidic channel residing between said first microfluidic channel and said fifth microfluidic channel;
a third lateral geometrical meniscus-pinning feature residing between said fourth microfluidic channel and said fifth microfluidic channel within said first portion of said fifth microfluidic channel, said third lateral geometrical meniscus-pinning feature being configured to resist fluid flow between said fifth microfluidic channel and said fourth microfluidic channel within said first portion of said fifth microfluidic channel;
a sixth microfluidic channel extending within said second horizontal planar region, said sixth microfluidic channel residing vertically adjacent to said fifth microfluidic channel along a second portion of said fifth microfluidic channel such that said sixth microfluidic channel is in direct vertical fluid communication with said fifth microfluidic channel along said second portion of said fifth microfluidic channel in the absence of a membrane therebetween; and
a second vertical geometrical meniscus-pinning feature residing between said sixth microfluidic channel and said fifth microfluidic channel within said second portion of said fifth microfluidic channel, said vertical geometrical meniscus-pinning feature being configured to resist fluid flow between said fifth microfluidic channel and said sixth microfluidic channel within said second portion of said fifth microfluidic channel.

15. The microfluidic device according to claim 14 wherein said first portion of said fifth microfluidic channel overlaps at least in part with said second portion of said fifth microfluidic channel.

16. The microfluidic device according to claim 14 further comprising: a seventh microfluidic channel extending within said first horizontal planar region, said seventh microfluidic channel residing laterally adjacent to said fifth microfluidic channel along a third portion of said fifth microfluidic channel, such that said seventh microfluidic channel is in direct lateral fluid communication with said fifth microfluidic channel along said third portion of said fifth microfluidic channel in the absence of a membrane therebetween, said fifth microfluidic channel residing between said fourth microfluidic channel and said seventh microfluidic channel; and
a fourth lateral geometrical meniscus-pinning feature residing between said seventh microfluidic channel and said fifth microfluidic channel within said third portion of said fifth microfluidic channel, said fourth lateral geometrical meniscus-pinning feature being configured to resist fluid flow between said fifth microfluidic channel and said seventh microfluidic channel within said third portion of said fifth microfluidic channel.

17. The microfluidic device according to claim 1 wherein a substrate of said microfluidic device comprises a planar surface residing below said first horizontal planar region, such that said planar surface and said second horizontal planar region reside on opposite sides of said first horizontal planar region, and wherein a portion of said substrate residing between said planar surface and said first microfluidic channel is sufficiently transparent to permit microscopic imaging of at least said first microfluidic channel.

18. The microfluidic device according to claim 1 wherein said first microfluidic channel comprises a gel, and wherein said second microfluidic channel and said third microfluidic channel are substantially absent of said gel.

19. The microfluidic device according to claim 1 wherein said second microfluidic channel and said third microfluidic channel each comprise a gel, and wherein said first microfluidic channel is substantially absent of said gel.

20. A microfluidic apparatus comprising:
a plurality of microfluidic devices according to claim 1, wherein each microfluidic device is defined within a common substrate.

21. The microfluidic apparatus according to claim 20, wherein said plurality of microfluidic devices are defined in an arrayed format on a fluidic chip.

22. The microfluidic apparatus according to claim 21 wherein said fluidic chip has a length of 75 mm plus or minus 2 mm and a width of 26 mm plus or minus 2 mm, such that lateral dimensions of said fluidic chip are substantially equivalent to those of a conventional microscope slide.

23. The microfluidic apparatus according to claim 20 wherein said plurality of microfluidic devices are defined in an arrayed format on a microplate.

24. A microfluidic apparatus comprising:
a plurality of microfluidic devices according to claim 1, wherein each microfluidic device is defined within a common substrate; and
wherein, for at least one adjacent pair of microfluidic devices, said adjacent pair of microfluidic devices comprising a first microfluidic device and a second microfluidic device:
said first microfluidic channel of said first microfluidic device resides laterally adjacent to said second microfluidic channel of said second microfluidic device along an additional portion of said first microfluidic channel, such that said second microfluidic channel of said second microfluidic device is in direct lateral fluid communication with said first microfluidic channel of said first microfluidic device along said first portion of said first microfluidic channel of said first microfluidic device in the absence of a membrane therebetween; and
an additional lateral geometrical meniscus-pinning feature resides between said second microfluidic channel of said second microfluidic device and said first microfluidic channel of said first microfluidic device within said additional portion of said first microfluidic channel, said lateral geometrical meniscus-pinning feature being configured to resist fluid flow between said first microfluidic channel of said first microfluidic device and said second microfluidic channel of said second microfluidic device within said additional portion of said first microfluidic channel of said first microfluidic device.

25. The microfluidic apparatus according to claim 24 wherein said plurality of microfluidic devices are defined in an arrayed format on a fluidic chip.

26. The microfluidic apparatus according to claim 25 wherein said fluidic chip has a length of 75 plus or minus 2 mm and a width of 26 plus or minus 2 mm, such that lateral dimensions of said fluidic chip are substantially equivalent to those of a conventional microscope slide.

27. The microfluidic apparatus according to claim 24 wherein said plurality of microfluidic devices are defined in an arrayed format on a microplate.

28. A method of performing microfluidic perfusion of a cell-containing structure, the method comprising:
providing a microfluidic device according to claim 1;
forming the cell-containing structure within the first microfluidic channel such that the cell-containing structure contains viable cells; and
injecting a perfusion liquid into one or both of the second microfluidic channel and the third microfluidic channel, the perfusion liquid comprising growth media; and
incubating the microfluidic device while contacting the perfusion liquid with the cell-containing structure.

29. The method according to claim 28 further wherein the perfusion liquid is delivered to both the second microfluidic channel and the third microfluidic channel to perform perfusion of the cell-containing structure from both lateral and vertical directions, thereby facilitating diffusion and/or advection between the first microfluidic channel and the second microfluidic channel, and facilitating diffusion and/or advection between the first microfluidic channel and the third microfluidic channel.

30. The method according to claim 28 further comprising:
collecting the perfusion liquid after having contacted the perfusion liquid with the cell-containing structure, thereby obtaining collected perfusion liquid; and
detecting at least one secreted factor in the collected perfusion liquid, the secreted factor having been secreted by the viable cells within the cell-containing structure during perfusion.

31. The method according to claim 28 wherein the perfusion liquid is delivered to one of the second microfluidic channel and the third microfluidic channel, and wherein the other of the second microfluidic channel and the third microfluidic channel is employed to collect one or more secreted factors secreted by the viable cells within the cell-containing structure.

32. The method according to claim 31 further comprising detecting at least one of the one or more secreted factors during perfusion of the cell-containing structure.

33. The method according to claim 28 wherein the cell-containing structure is formed by:
injecting a precursor liquid into said first microfluidic channel, the precursor liquid comprising the viable cells, the precursor liquid being confined within said first microfluidic channel by the lateral geometrical meniscus-pinning feature and the vertical geometrical meniscus-pinning feature; and
hardening the precursor liquid to form the cell-containing structure within the first microfluidic channel.

34. The method according to claim 28 wherein the cell-containing structure is formed by:
injecting a precursor liquid into the first microfluidic channel, the precursor liquid being confined within said first microfluidic channel by the lateral geometrical meniscus-pinning feature and the vertical geometrical meniscus-pinning feature; and
hardening the precursor liquid to form a gel structure within the first microfluidic channel;
injecting a cell-containing liquid into at least one of said second microfluidic channel and said third microfluidic channel, the cell-containing liquid comprising the viable cells; and
incubating the microfluidic device to facilitate perfusion of the gel structure with the viable cells, thereby forming the cell-containing structure.

35. The method according to claim 28 further comprising imaging the cell-containing structure.

36. The method according to claim 35 wherein the substrate comprises a planar surface residing below the first horizontal planar region, such that the planar surface and the second horizontal planar region reside on opposite sides of the horizontal planar region, and wherein a portion of the substrate residing between the planar surface and the first microfluidic channel is substantially transparent, the method further comprising:
imaging the cell-containing structure through the planar surface.

37. The method according to claim 35 wherein the imaging is performed during perfusion of the cell-containing structure.

38. A method of performing microfluidic perfusion of cell-containing structures, the method comprising:
providing a microfluidic device according to claim 1;
forming cell-containing structures within the second microfluidic channel and the third microfluidic channel such that the cell-containing structures contain viable cells; and
injecting a perfusion liquid into the first microfluidic channel, the perfusion liquid comprising growth media; and
incubating the microfluidic device while contacting the perfusion liquid with the cell-containing structures.

39. The method according to claim 38 further comprising:
collecting the perfusion liquid after having contacted the perfusion liquid with the cell-containing structure, thereby obtaining collected perfusion liquid; and
detecting at least one secreted factor in the collected perfusion liquid, the secreted factor having been secreted by the viable cells within the cell-containing structure during perfusion.

40. The method according to claim 38 further comprising detecting at least one of the one or more secreted factors during perfusion of the cell-containing structure.

41. The method according to claim 38 wherein the cell-containing structures are formed by:
injecting a precursor liquid into the second microfluidic channel and the third microfluidic channel, the precursor liquid comprising the viable cells, the precursor liquid being confined within the second microfluidic channel by the lateral geometrical meniscus-pinning feature, and the precursor liquid being confined within the third microfluidic channel by the vertical geometrical meniscus-pinning feature; and
hardening the precursor liquid to form the cell-containing structure within the second microfluidic channel and the third microfluidic channel.

42. The method according to claim 38 wherein the cell-containing structures are formed by:
injecting a precursor liquid into the second microfluidic channel and the third microfluidic channel, the precursor liquid being confined within the second microfluidic channel by the lateral geometrical meniscus-pinning feature, and the precursor liquid being confined within the third microfluidic channel by the vertical geometrical meniscus-pinning feature; and
hardening the precursor liquid to form a gel structure within the second and third microfluidic channels;
injecting a cell-containing liquid into the first microfluidic, the cell-containing liquid comprising the viable cells; and
incubating the microfluidic device to facilitate perfusion of the gel structure with the viable cells, thereby forming the cell-containing structure.

43. The method according to claim 38 further comprising imaging the cell-containing structure.

44. The method according to claim 43 wherein the imaging is performed during perfusion of the cell-containing structure.
